(19) United States
(12) Patent Application Publication
(10) Pub. No.: US 2010/0221700 A1
(43) Pub. Date: Sep. 2, 2010

(54) METHOD OF MONITORING HIV INFECTION

(76) Inventors: Barton F. Haynes, Durham, NC (US); Nancy G. Smith, Durham, NC (US)

Correspondence Address: NIXON & VANDERHYE, PC, 901 NORTH GLEBE ROAD, 11TH FLOOR, ARLINGTON, VA, 22203, US

(21) Appl. No.: 12/448,808

(22) PCT Filed: Jan. 11, 2008
(86) PCT No.: PCT/US08/00412

§ 371 Date: Mar. 23, 2010

Related U.S. Application Data
(60) Provisional application No. 60/879,803, filed on Jan. 11, 2007.

(51) Int. Cl. C12Q 1/70

(52) U.S. Cl. 435/5

(57) ABSTRACT

The present invention relates, in general, to human immunodeficiency virus (HIV) and, in particular, to a method of monitoring the intensity of HIV infection and predicting the time to progression to acquired immunodeficiency syndrome (AIDS).

This application claims priority from Provisional Application No. 60/879,803, filed Jan. 11, 2007, the entire content of which is incorporated by reference.

This invention was made with government support under Grant No. 1 UO1 AI0678501 awarded by the National Institutes of Health. The government has certain rights in the invention.

### TECHNICAL FIELD

The present invention relates, in general, to human immunodeficiency virus (HIV) and, in particular, to a method of monitoring the intensity of HIV infection and predicting the time to progression to acquired immunodeficiency syndrome (AIDS).

### BACKGROUND

The time of appearance of antibodies in the development of acute HIV infection has been recently mapped and it has been shown that most of the antibodies arise after a delay in the peak response to HIV envelope epitopes of approximately two to three weeks. Indeed, the most protective antibodies, those that neutralize autologous virus, can be delayed for up to a year (Wei et al, Nature 422:307-12 (2003); Richman et al, Proc. Natl. Acad. Sci. USA 100:4144-9 (2003)) (FIG. 1).

Fiebig et al (AIDS 17:1871-1879 (2003)) have studied plasma panels from plasma donors in US Blood Banks and have found that the plasma panels represent the earliest time points sampled surrounding HIV transmission (FIG. 2). The time course of these panels begins before any detectable virus is present, and then continues through the viral ramp-up stages, or Eclipse phase, through the first and second states of HIV, when seroconversion has not yet occurred. FIG. 3 shows the viral loads of 11 such panels of plasma.

To begin to understand the “delay” in induction of antibodies at the time of HIV transmission, the first question to be addressed was whether there are immunosuppressive events, such as massive apoptosis, with release of phosphatidylserine microparticles at the time of viral load ramp up during acute HIV infection (Mattapallil et al, Nature 434:1093 (2005), Veazey et al, Science 280:427 (1998), Guadalupe et al, J. Virol. 77:11708 (2003), Benchley et al, J. Exp. Med. 200:749 (2004), Mehandru et al, J. Exp. Med. 200:761 (2004), Esser et al, J. Virol. 75: 6173-6182 (2001), Aupelx et al, J. Clin Invest. 99:1546-1554 (1997), Callahan et al, J. Immunol. 170:4840-4845 (2003)). Apoptotic microparticles are the products of either activated or apoptotic cells, that are increased in the plasma of a number of diseases, including autoimmune diseases such as systemic lupus erythematosus and rheumatoid arthritis (Distler et al, Arth. Rheum. 52:33337-3348 (2005), Tesse et al, Arterioscler. Thromb. Vasc. Biol. 25:2522-2527 (2005), Cerri et al, J. Immunol. 177:1975-1980 (2006)), Crohn's disease (Chamouard et al, Dig. Dis. Sci. 50:574-580 (2005)), coronary artery disease and other forms of heart disease (Boulanger et al, Cardiovas. Res. 67:1-3 (2005)), and chronic HIV-1 infection (Esser et al, J. Virol. 75:6173-6182 (2001), Aupelx et al, J. Clin Invest. 99:1546-1554 (1997)). Apoptotic microparticles can bind to non-apoptotic cells and induce apoptosis (Distler et al, Apoptosis 10:731-741 (2005)), are procoagulant (Distiller et al, Apoptosis 10:731-741 (2005)), pro-inflammatory (Tesse et al, Arterioscler. Thromb. Vasc. Biol. 25:2522-2527 (2005), Cerri et al, J. Immunol. 177:1975-1980 (2006)), and can be immunosuppressive for T and B cell responses to specific antigen (Esser et al, J. Virol. 75:6173-6182 (2001), Fadok et al. J. Immunol. 174:1393 (2005)).

Microparticle levels correlate with the levels of IL-6 in healthy adults (Chirinos et al, Amer. J. Card. 95:1258-1260 (2005)), are increased in acute coronary syndromes and correlate with severity of angiographic coronary lesions (reviewed in Mezentsev, Am. J. Physiol. Heart Circ. Physiol. 289:H1106-H1114 (2005)). CD31/annexin V+apoptotoc microparticles correlate with coronary endothelial function in patients with coronary artery disease (Werner et al, Arterioscler. Throm. Vasc. Biol. 26:112-116 (2006), Epub October 2005). Aupelx et al (J. Clin Invest. 99:1546-1554 (1997)) have suggested that measuring levels of apoptotic microparticles in plasma may provide information regarding the severity of immune cell destruction in HIV, and also provide an indicator of the responsivness to anti-retroviral drugs.

The present invention provides a method of a method of predicting the course of HIV infection in a patient during acute HIV infection (AHI), a method of determining the degree of potential damage to the immune system in AHI, a method of determining the need for anti-retroviral treatment in AHI and a method of monitoring the course of that infection by measuring plasma levels of microparticles coupled with tests of cell activation and/or apoptosis.

### SUMMARY OF THE INVENTION

The present invention relates generally to HIV. More specifically, the invention relates to a method of monitoring the intensity of HIV infection and predicting the time to progression to AIDS.

Objects and advantages of the present invention will be clear from the description that follows.

### BRIEF DESCRIPTION OF THE DRAWINGS

FIG. 1. Summary of antibody responses immediately following acute HIV-1 infection. Samples from plasma donors at various time points before, during, and after acute HIV-1 infection (AHI) were assayed for the presence of antibodies against gp140, V3 loop, the CD4 Binding Site (BS) the membrane proximal external region (MPER) by ELISA. Samples were also assayed for the presence of neutralizing antibodies (Nab) and the presence of 2F5, 4E10, and 2G12 neutralizing antibodies. HIV RNA was quantified using the bDNA technique (Chiron Diagnostics).

FIG. 2. A schematic, semi-quantitative display of the progression of HIV markers (adapted from Fiebig et al, AIDS17:1871 (2003)).

FIG. 3. Viral loads of plasma panels. Plasma from blood bank donors drawn at various time points before, during, and after HIV-1 infection were assayed for viral RNA using the bDNA technique (Chiron Diagnostics).

FIGS. 4A-4C. Soluble Fas Ligand levels during AHI. Plasma from blood bank donors drawn at time points before, during, and after HIV-1 transmission were assayed for the presence of soluble Fas Ligand by ELISA (Diaclone). Each specimen was assayed in duplicate, and error bars represent standard deviation. Viral load was also measured for these panels (bDNA, Chiron diagnostics) and is displayed on the secondary axis (copies/ml). Three representative panels are shown.

FIG. 5. Percent increase in Fas Ligand levels before and after Day 0. For each individual panel member, the average Fas Ligand level up to and including Day 0 was compared with the average Fas Ligand level after Day 0.

FIGS. 6A-6C. Tumor Necrosis Factor Receptor 2 (TNFR2) levels during AHI. Plasma from blood bank donors drawn at time points before, during, and after HIV-1 infection were assayed for the presence of soluble TNFR2 by ELISA (Diaclone). Each specimen was assayed in duplicate, and error bars represent standard deviation. Viral load was also measured for these panels (bDNA, Chiron Diagnostics) and is displayed on the secondary axis (copies/1M. Three representative panels are shown.

FIG. 7. Percent increase in TNFR2, levels before and after Day 0. For each individual panel member, the average TNFR2 level up to and including Day 0 was compared with the average TNFR2 level after Day 0.

FIGS. 8A-8C. TNF-related apoptosis inducing ligand (TRAIL), levels during AHI. Plasma from blood bank donors drawn at time points before, during, and after HIV-1 infection were assayed for the presence of soluble TRAIL by ELISA (Hycult). Each specimen was assayed in duplicate, and error bars represent standard deviation. Viral load was also measured for these panels (bDNA, Chiron Diagnostics) and is displayed on the secondary axis, (copies/ml). Three representative panels are shown.

FIG. 9. Percent increase in TRAIL levels before and after Day 0. For each individual panel member, the average TRAIL level up to and including Day 0 was compared with the average TRAIL level after Day 0.

FIG. 10. TRAIL levels for Blood Bank Panel 6246. Plasma specimens from panel 6246 were assayed for soluble TRAIL levels by ELISA (Diaclone), as well as for the presence of HIV RNA (bDNA, Chiron Diagnostics) (copies/ml). Specimen 6246-15 (11 days after the first time point of viral load>100 copies/ml) was chosen for flow cytometry analysis for microparticles.

FIG. 11. Flow cytometry analysis of purified microparticles in AHI plasma. Purified microparticles were prepared by stimulating Jurkat cells (ATCC TIB-152) with staurosporine for 24 hours. Microparticles were harvested by high-speed centrifugation, and pellets were resuspended in PBS. Plasma (specimen 6246-15) was diluted in PBS. All samples were analyzed at a final dilution of 1:10 in PBS. Gating was determined by the PBS sample, and total events were recorded for 2 minutes.

FIG. 12. Phenotypic analysis of purified microparticles and AHI plasma. A purified microparticle preparation or plasma (specimen 6246-15) was diluted and stained directly with APC-conjugated antibodies against CD3 and CD45. Gating was determined by the PBS sample, and total events, (#e), were recorded for 2 minutes. The percentages displayed represent the events within the gate, as well as mean fluorescent intensity, (MFI), and the signal to noise ratio, (S/N).

FIG. 13. Anti-phosphatidylserine staining of microparticles. Plasma (specimen 6246-15) was stained with the 2aG4 antibody against phosphatidylserine or the C44 control antibody. The samples were then incubated with a secondary goat anti-human FITC-conjugated antibody.

FIG. 14. HIV-infected T cells display phosphatidylserine. H-9 cells (ATCC CRL-8543) were either infected with HIV or remained uninfected as a control. Cells were then stained with a control antibody, (anti-human epidermal grown factor receptor, Erbitux), or anti-phosphatidylserine, (Tarvicin). Cells were then incubated with a secondary conjugated (FITC- or gold-labeled) goat anti-human antibody.

FIG. 15. Microparticles expressing phosphatidylserine in AHI Plasma. Various time points of panel 6246 (21 days before viral load was detected, day −21, and 7, 11 and 14 days after viral load was measured to be >100 copies/ml) were assayed by flow cytometry for the presence of microparticles stained with anti-phosphatidylserine antibodies (2aG4). As a control, plasma from a normal, uninfected blood bank donor was also assayed in the same manner.

FIGS. 16A-16C. Comparison with HBV panels.

FIGS. 17A-17C. FIG. 17A. TEM of microvesicles isolated from OT1 T cells. For immuno-micrograph studies, OT1 microvesicles were applied to poly-L-lysine coated electron micrograph grids, blocked with 1% BSA, and dual labeled with anti-CD8a and anti-TCRb (FIG. 17B) antibodies. Either 15 nm (CD8, thick arrows) or 5 nm (TCR, thin arrows) Au-labeled streptavidin conjugated antibodies were used as secondary antibodies. Grids were washed and treated with OsO₄ and examined using transmission electron microscopy. FIG. 17C. A schematic of the BIAcore binding assay for anchoring microvesicles on L1 sensor chip (via a lipophilic linker) and the binding interactions with peptide-MHC complexes.

FIG. 18. OT1 microvesicles were anchored on BIAcore L1 sensor chip as shown in the schematics in FIG. 17C. Following stabilization of baseline and injection of BSA to block non-specific binding, Ova-K^{b} (upper line) or control VSV-K^{b} (lower line) tetramers were injected at 0.25 mg/mL. Binding responses show peptide specific binding of OVA-K^{b} to OT1 TCR expressing microvesicles.

FIGS. 19A-19C. Plasma viral loads of HIV-1, Hepatitis C Virus, (HCV), and Hepatitis B Virus, (HBV), plasma donor subjects. Thirty HIV+seroconversion plasma donor plasma panels (HBV and HCV negative), ten HBV plasma donor seroconversion panels (HIV negative), and 10 HCV plasma donor seroconversion panels (HIV negative), were studied. Panels demonstrate the kinetics of viral load ramp-up in (FIG. 19A) HIV, (FIG. 19B) HCV, and (FIG. 19C) HBV. T₀ was determined to be the first day that the viral load reached 100 copies/ml for HIV, 600 copies/ml for HCV, and 700 copies/ml for HBV.

FIGS. 20-20C. Plasma Markers of Cell Death. FIG. 20A. TRAIL, TNFR2, and Fas Ligand were measured for each plasma sample by ELISA and compared to viral load levels. Three representative subjects are shown. FIG. 20B. In order to compare increases in plasma markers of apoptosis between subjects, the mean before T₀ was compared to the mean after T₀, and percent increases were calculated. FIG. 20C. The same plasma markers of apoptosis were also measured in 10 HCV or HBV acutely infected subjects. Representative results in one HCV and one HBV subject are shown.

FIGS. 21A-21C. Comparison of Peak Plasma Analytes Levels With the First Plasma in Each Panel, and with Uninfected Plasma. FIG. 21A. Boxplot analyses were performed for each group of data, and the results of the acute HIV-1, HBV and HCV panels are displayed, with vertical lines signifying the maximum and minimum values. The P values were computed with a Student's T test. Shaded boxes indicate p<0.01. FIG. 21B. Within the 30 acute HIV-1 infected patients studied, 30/30, 27/30, 26/30 demonstrated TRAIL, TNFR2, and Fas Ligand level respectively, peaks near (within 15 days) the peak viral load. Furthermore, 21/30 patients demonstrated TRAIL level peaks before the viral load peaked, while 16/30 TNFR2 and Fas Ligand level peaks coincident with the peak in viral load. The same analysis was performed for the 10 HCV and HBV subjects studied. FIG. 21C show timing of peak analyte relative to maximum viral expansion. Results are from a paired Wilcoxon rank test, and a low p value indicates that the two means (of the peak dates of interest) are significantly different. This implies that the mean ‘arrival times’ of the peaks (e.g., peak expansion day and peak TRAIL day) are significantly different. The delay between the arrival times can be described in terms of a mean, a median, and an interquartile range. The arrival time of each analyte maximum is compared with the time of peak viral expansion (left-most box). A p value arising from the Wilcoxon test is shown above the analyte of interest. The significant p values in indicate that the average day of peak analyte level was significantly different from the average day of peak or maximal rate of viral expansion. Also noted are mean delay times (median times in parentheses). Open circles indicate outlier values.

FIGS. 22A and 22B. Relative microparticle counts in plasma samples. FIG. 22A. Relative microparticle counts were acquired for each sequential time point for each plasma donor subject. From 30 subjects studied, three representative subjects are shown. FIG. 22B. The same analysis was performed for 10 HBV and HCV infected subjects. The results of sequential time points in one representative HCV and one HBV subject are shown. Microparticle (MP) count is ♦ and viral load is x.

FIGS. 23A-23C. Morphology and CCR5 Expression of Plasma MP in Acute HIV-1 Infection. FIG. 23A shows electron micrograph of plasma MP harvested from an acute HIV-1 infected subject (6244). Plasma MP were pelleted by ultracentrifugation and purified over a sucrose pad. Large arrowheads show double membrane microparticles 100 nM to 1 μM in size. Small arrowheads show 30-100 nm particles (exosomes). Bar=100 nm. FIG. 23B shows CCR5+ MP were present during acute HIV-1 infection. FIG. 23B. Flow cytometric analysis of MP that are CCR5+ (lower panel) compared to the isotype negative control (upper panel). FIG. 23C shows comparison of the number of CCR5+ MP (% CCR5+ MP determined by phenotypic flow analyses multiplied by the relative MP count) in 5 different seroconversion panels, in the first plasma sample and at peak MP counts.

FIGS. 24A and 24B. MP-Induced suppression of PWM/oCpG-stimulated tonsil B cells. FIG. 24A. Tonsil cells derived from healthy donors were cultured alone or in the presence of PWM and oCpG with or without PBMC-derived MP. The presence of MP induced reduced production of both total IgG and IgA. Data are representative of five experiments, and are presented as mean±SEM. FIG. 24B show a dose-dependent suppression of IgG by increasing amounts of PBMC MPs.

FIGS. 25A-25C. Development of flow cytometric techniques for measurement of plasma MP. A mixture of polystyrene beads was first assayed (FIG. 25A). Beads ranging from 0.1 μm to 1.0 μm in size were mixed in equal proportion, diluted, and analyzed with a BD LSRII. Side scatter was used as a size discriminator because of the enhanced ability of the photomultiplier tube to discriminate smaller particles than the diode of the forward scatter detector. To determine optimal dilution ranges, a series of serial dilutions of the polystyrene bead mixture was analyzed (FIG. 25B). It was found that any sample that was not sufficiently dilute yielded an event count that was falsely low due to coincidence and high abort rates. Dilution of sample to the point where only one particle flowed through the laser beam at a time allowed the event count processed by the cytometer to be more accurate. In fact, when the bead mixture was diluted at 1:1000, the 4 different sizes of beads could not be discriminated well whereas, at a 1:100,000 dilution, clear populations of each size could be detected. To analyze plasma microparticles (FIG. 25C), a similar dilution series was used to experimentally determine the optimal dilution (data not shown). The MP gate was drawn by including the 0.1 μm beads in the low side scatter range, and including the 1.0 μm beads in the higher side scatter range, while excluding particles that had very little forward and side scatter (see boxes in FIG. 25A and FIG. 25C). The polystyrene sizing beads were analyzed at a 1:100,000 dilution for each experiment, allowing all data to be gated in the same manner. In plasma samples, the majority of MP were found between 0.1 and 0.5 μm (the population within the microparticle gate that demonstrated side scatter area of less than 10⁴). Larger microparticles, greater than 0.5 μm but smaller than 1.0 μm were present, but were fewer in proportion.

FIG. 26. The effects of freeze/thaw cycles on the phenotype of plasma MP. Due to the low expression levels of some of the extracellular markers in the plasma donor samples, an investigation was made of the effects of freezing and thawing plasma on MP phenotype. Plasma from a HIV-1 chronically infected donor was divided into 2 aliquots. The first remained at 20° C. (fresh). The second was frozen/thawed 2×. All samples were then diluted, filtered, and centrifuged as described in Example 3. The MP resuspension was incubated with directly conjugated CD3, CD45, CD61 (a platelet MP marker), and Annexin V (that binds to phosphatidyl serine). The percentages within the boxes indicate the percentage of MP positive for that particular marker after background subtraction of the isotype controls. The percent CD3, CD4, CD61 and annexin positive MP was considerably decreased after two freeze/thaw cycles.

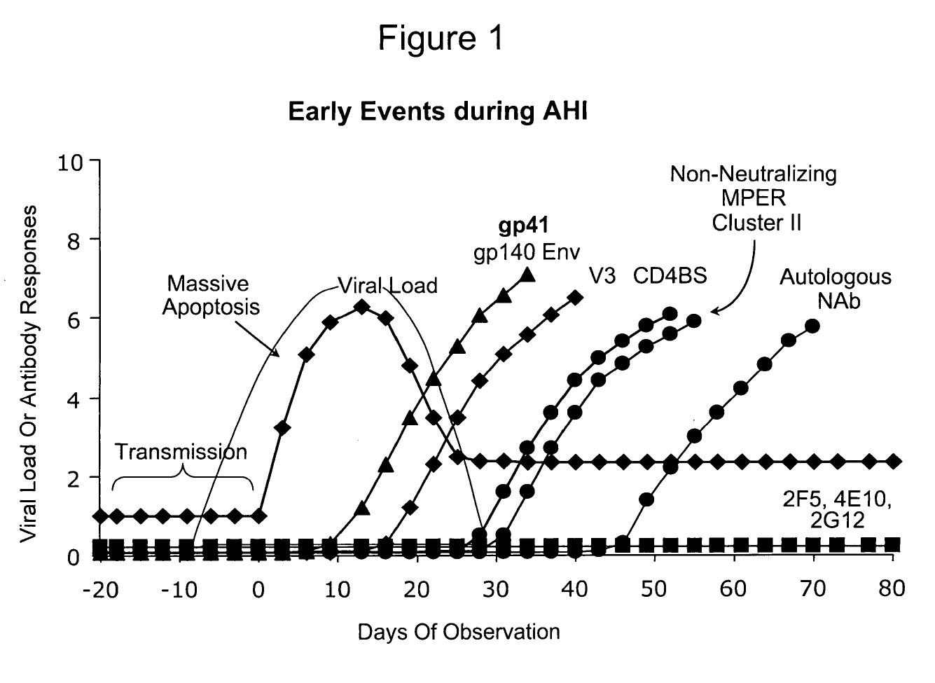

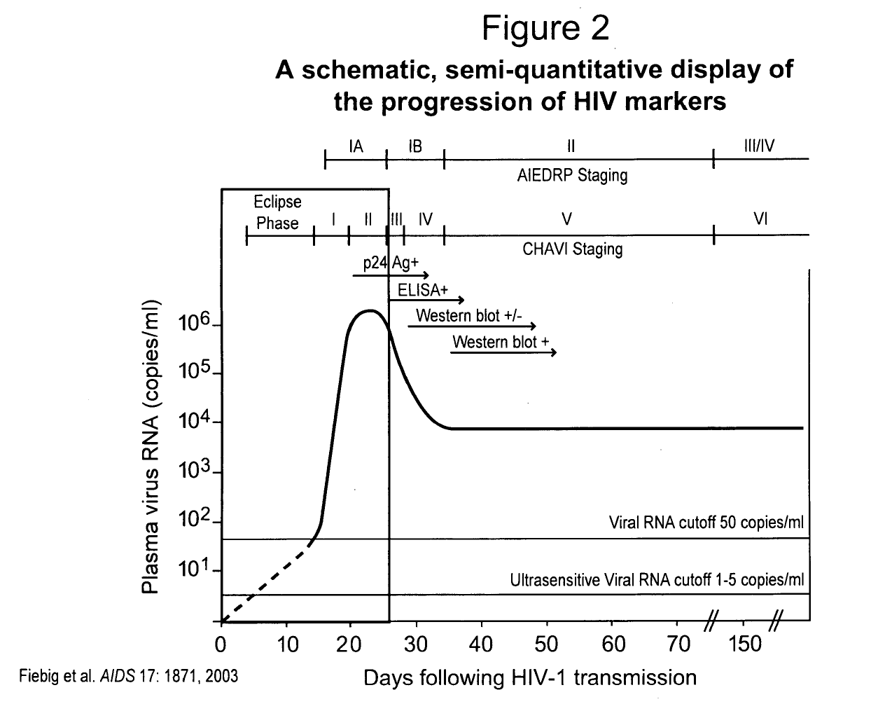

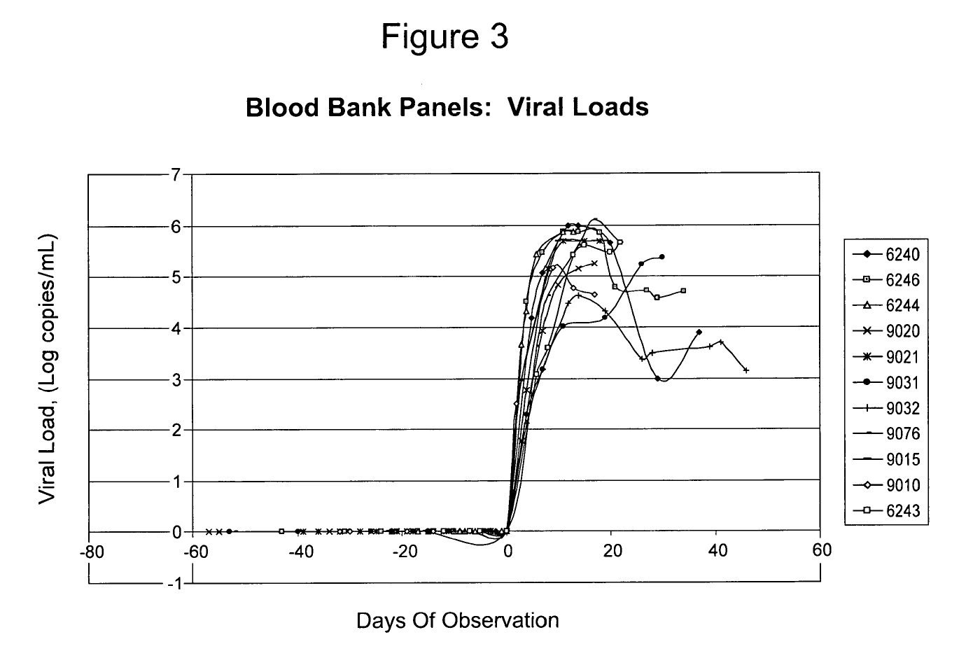

### DETAILED DESCRIPTION OF THE INVENTION

The present invention relates to methods of determining the degree of immune system destruction in HIV, of determining the prognosis and the course of the disease in AHI, and of determining the need for treatment in AHI. The present invention further relates to a method of monitoring the intensity of HIV infection and predicting the time to progression of AIDS. The methods are based on plasma tests of immune activation and/or apoptosis plus phenotypic and quantitative assays of plasma microparticles. In preferred embodiments, the methods comprise monitoring plasma levels of TRAIL, Fas Ligand and/or TNFR2. Other plasma markers of HIV-1 destruction of the immune system can be used either alone or in combination with these tests (one such marker is nuclear protein HMGB-1 that is released in plasma from apoptotic cells (Nowak et al, Cytokine 34:17-23 (2006), epub May 11, 2006)).

Microparticles can come from any immune or non-immune cells. Phenotypic characterization of microparticles has diagnostic use. HIV-1 virion microparticles are CD45− and thus CD45 can be used to distinguish virions from immune cell microparticles (Esser et al, Virol. 75:6173-6182 (2001)). In general, elevated T cell microparticles indicate destruction of T cells in vivo.

It is important to note that there is heterogeneity in the degree of increase in TNFR2 and Fas Ligand. This indicates that there are patients with massive immune system apoptosis and those with little immune system apoptosis at the time of AHI. Thus, identification of patients who need treatment with anti-HIV drugs (i.e., those with elevated Fas Ligand, TNFR2, microparticles and TRAIL levels) versus those who do not (no or minimal Fas Ligand, TNFR2, TRAIL and CD45, CD3 T cell microparticle levels) is important for directing treatment in AHI.

The data provided in the Examples that follow demonstrate that, at the time of viral load ramp-up in AHI, when a protective antibody needs to be made, there are high levels of microparticles, high TRAIL, high Fas Ligand, and high TNFR2 levels in plasma in many patients. That there is heterogeneity in the levels of these proteins and microparticles indicates that measurement of these parameters individually or in combination provides a prognostic test suitable for use in the setting of AHI to predict the course of HIV infection and time to AIDS. It will be appreciated that one or more of these parameters can also be considered with other markers of apoptosis and cell death and/or activation (e.g., soluble HMGB-1, CD25, CD69, or other soluble molecules of T cell activation/apoptosis).

While the details provided in the Examples below relate specifically to HIV, the invention includes within its scope methods of monitoring the progression and/or clinical course of other infectious diseases as well that are associated with apoptosis during acute infection (Bahl et al, J. Immunol. 176:4284-4295 (2006)) using these same parameters.

Certain aspects of the invention are described in greater detail in the non-limiting Examples that follows. This application is related to U.S. Prov. Appln. No. 60/859,496, filed Nov. 17, 2006, the entire content of which is incorporated herein by reference.

### Example 1

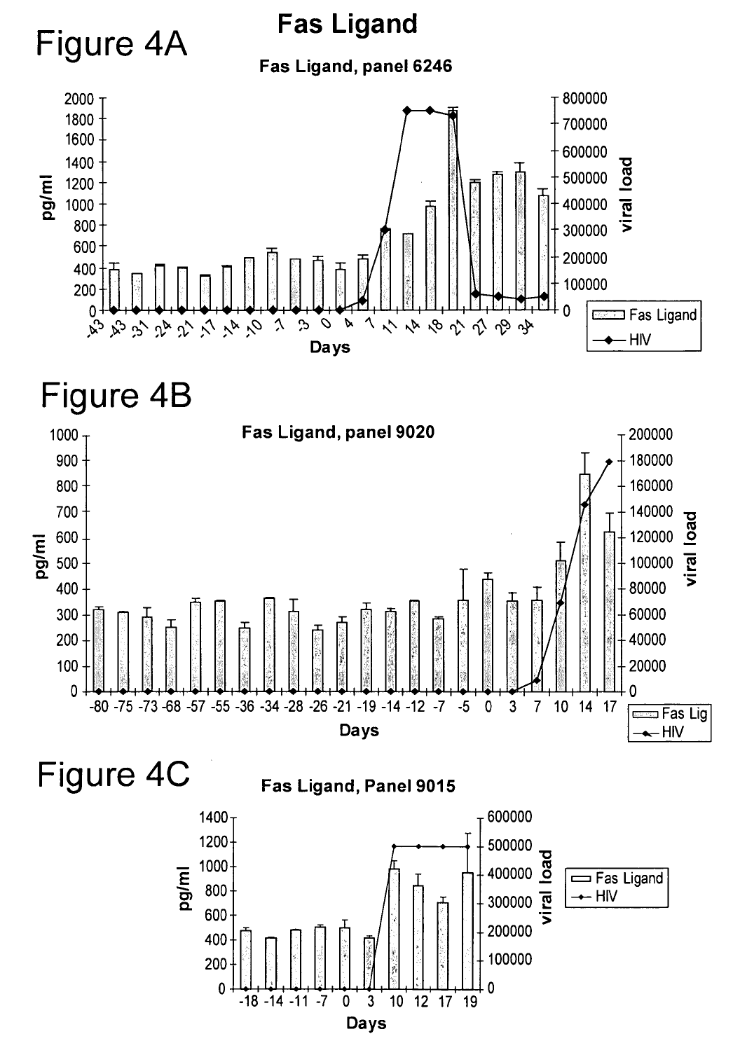

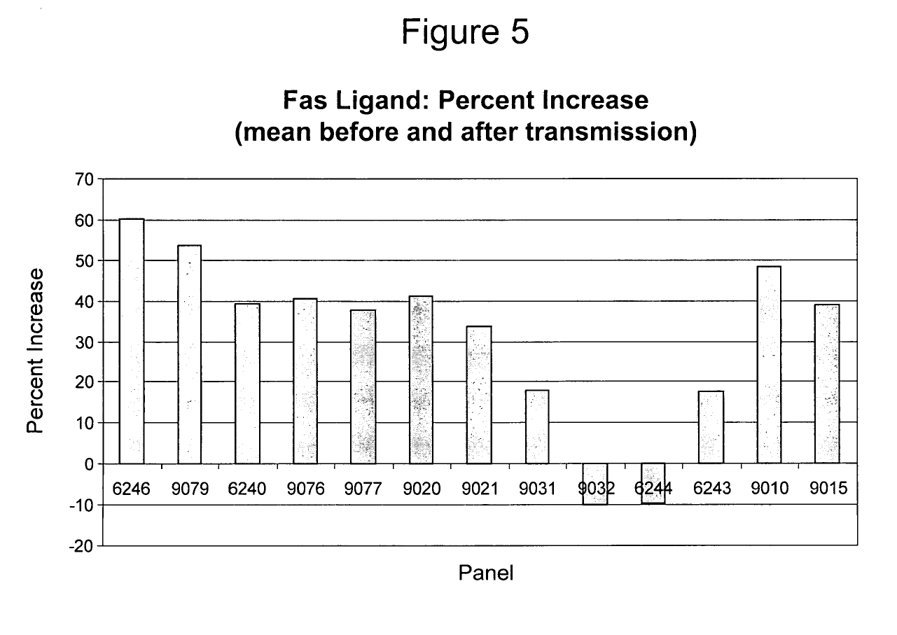

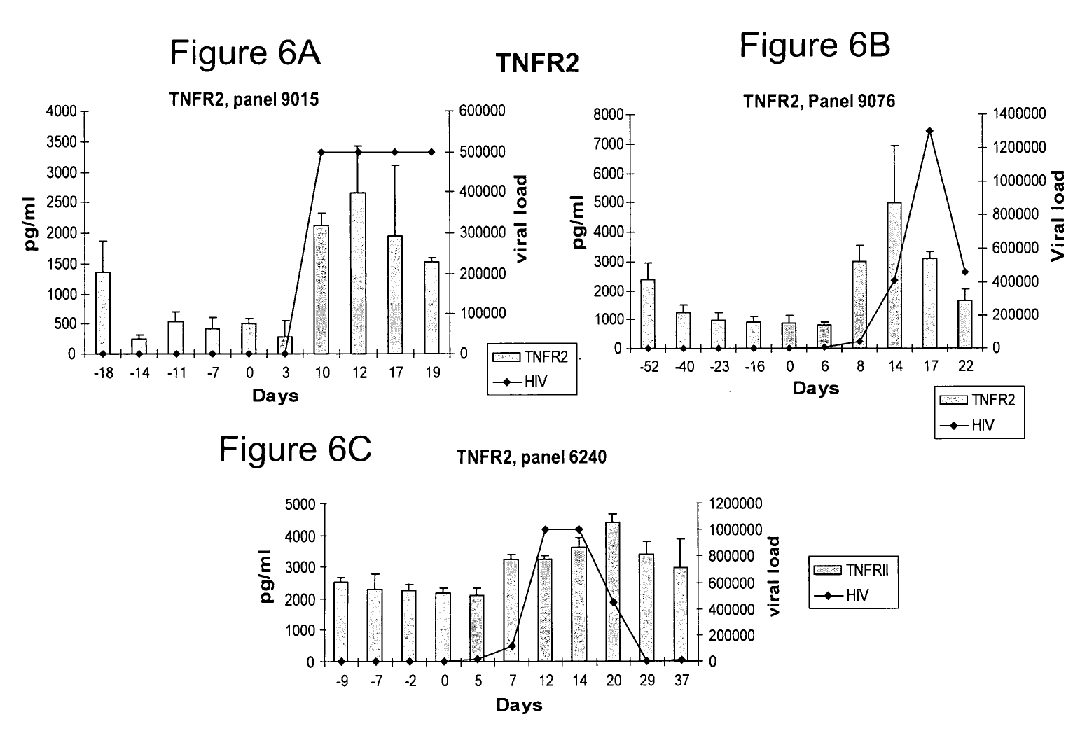

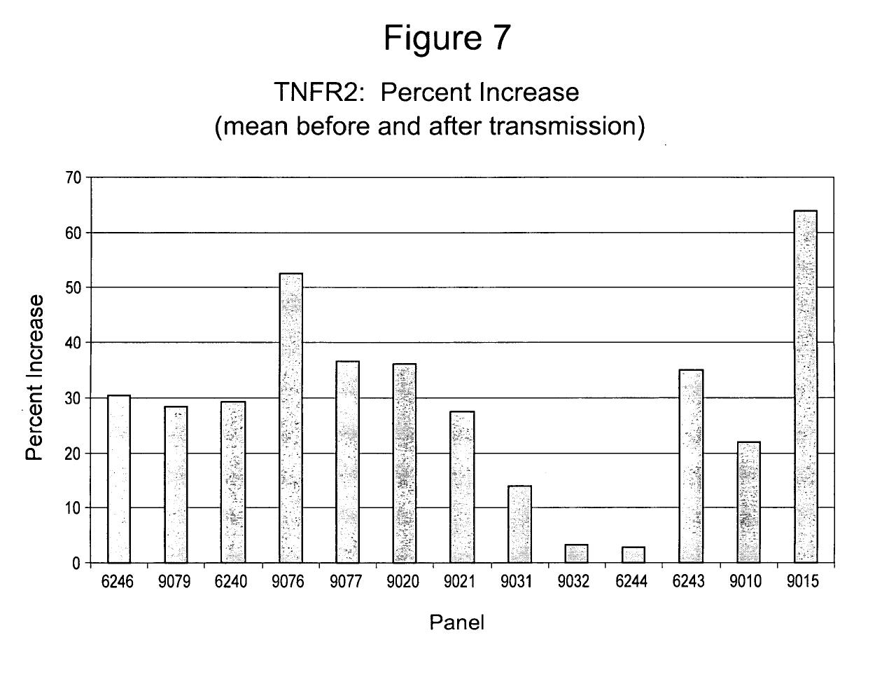

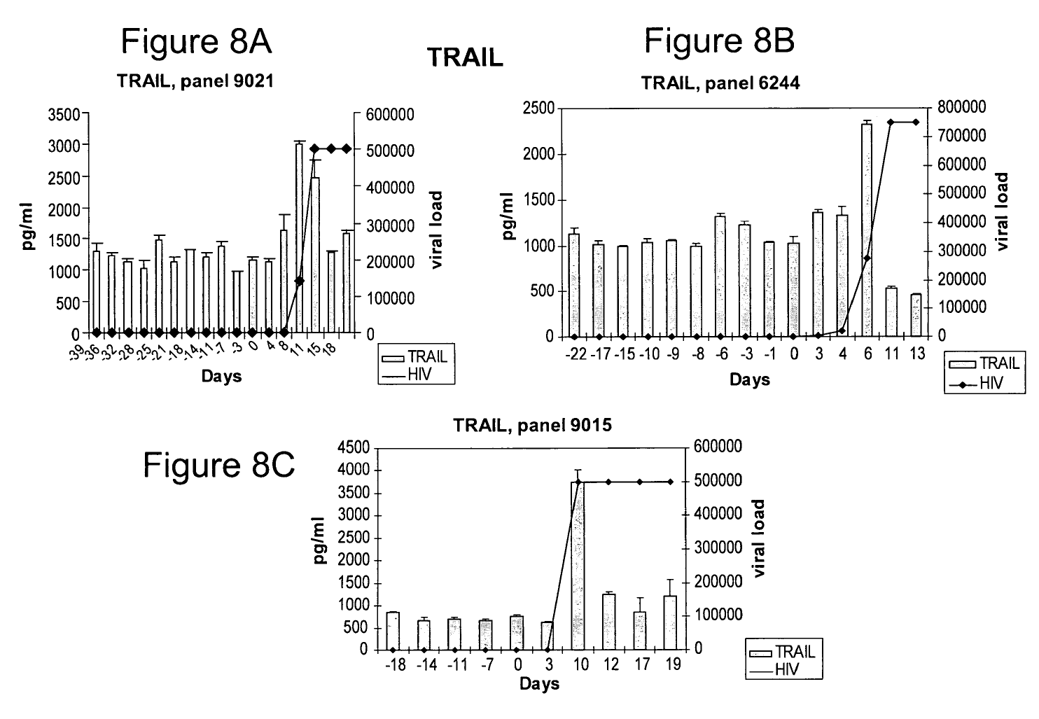

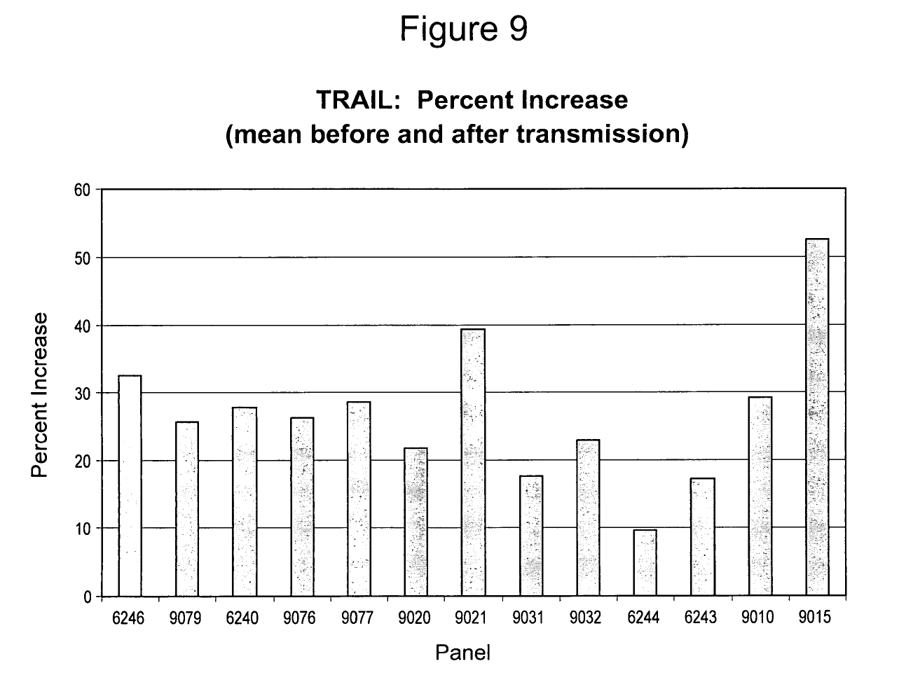

Several plasma markers of apoptosis have been studied in Blood Bank panels, including Fas Ligand, TNFR2, and TRAIL. TRAIL has been implicated in the activation induced cell death in HIV infection (Katsikis et al, J. Exp. Med. 186:1365-1372 (1997)). Fas Ligand has been found to be elevated in AHI (FIGS. 4 and 5). Elevations of Fas Ligand were observed in the plasma of 11/13 patients while it was not elevated in 2/13—implying no or less apoptosis in some patients and not others. Similarly, TNFR2 was elevated in 11/13 patients, and again, the same two patients with low Fas Ligand levels had no increases in plasma TNFR2 (FIGS. 6 and 7). Finally, TRAIL levels were elevated in all thirteen patients tested, with the two patients that were low in the other markers of apoptosis, low in TRAIL (FIGS. 8, 9).

Thus, the massive apoptosis that occurs with acute HIV infection with resulting release of TRAIL, mediation of apoptosis via FAS-FASL interactions, and release of PS containing viral and other particles, all conspire to initially immunosuppress the host, preventing rapid protective B cell responses.

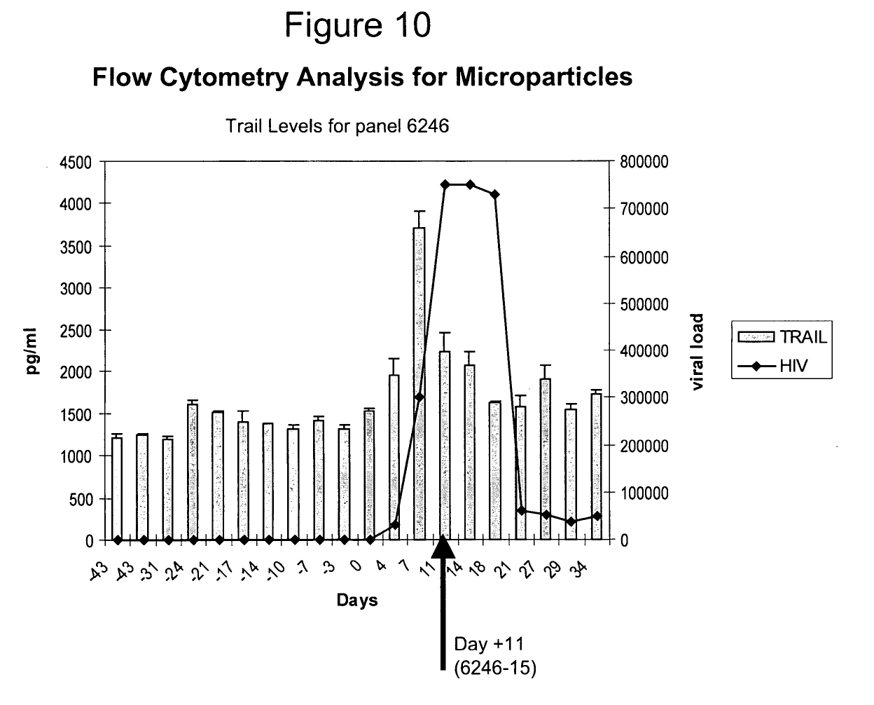

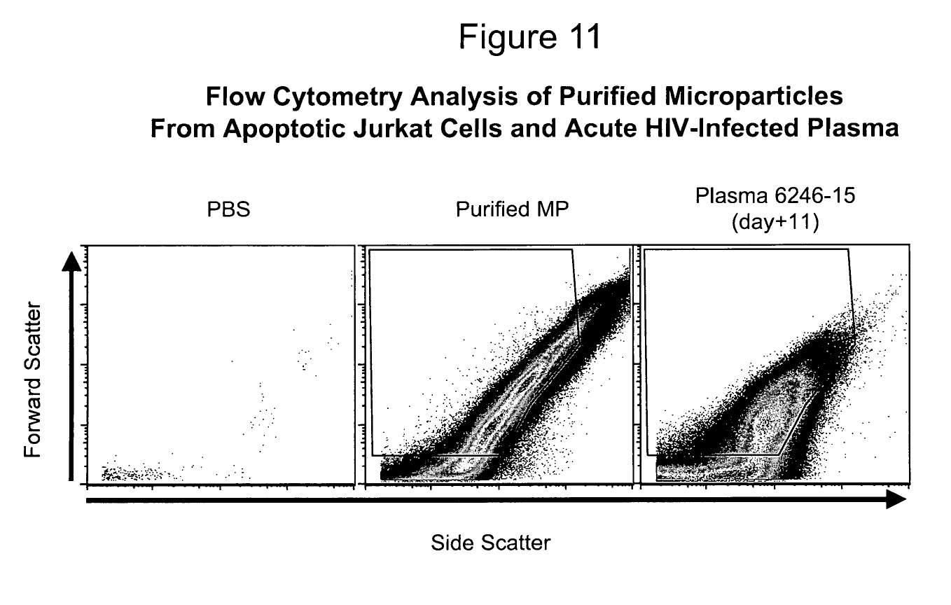

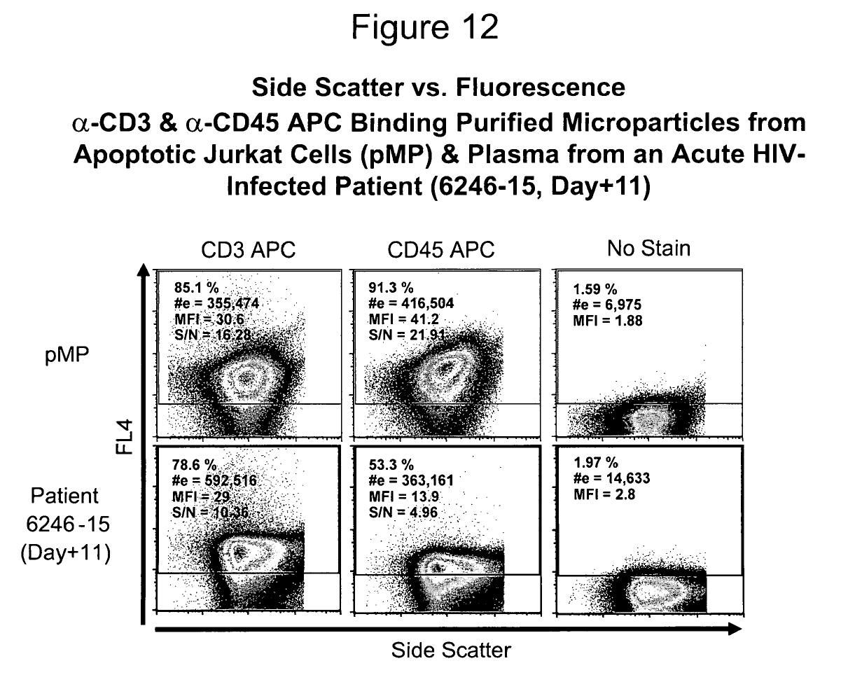

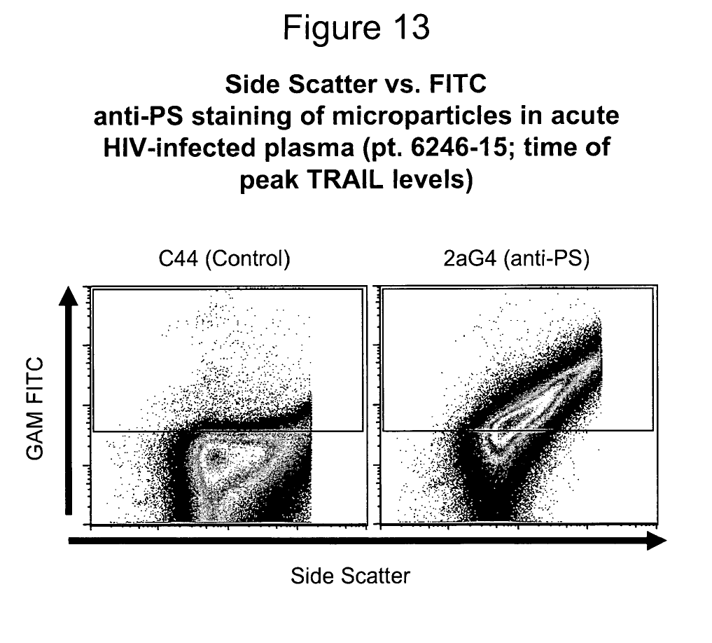

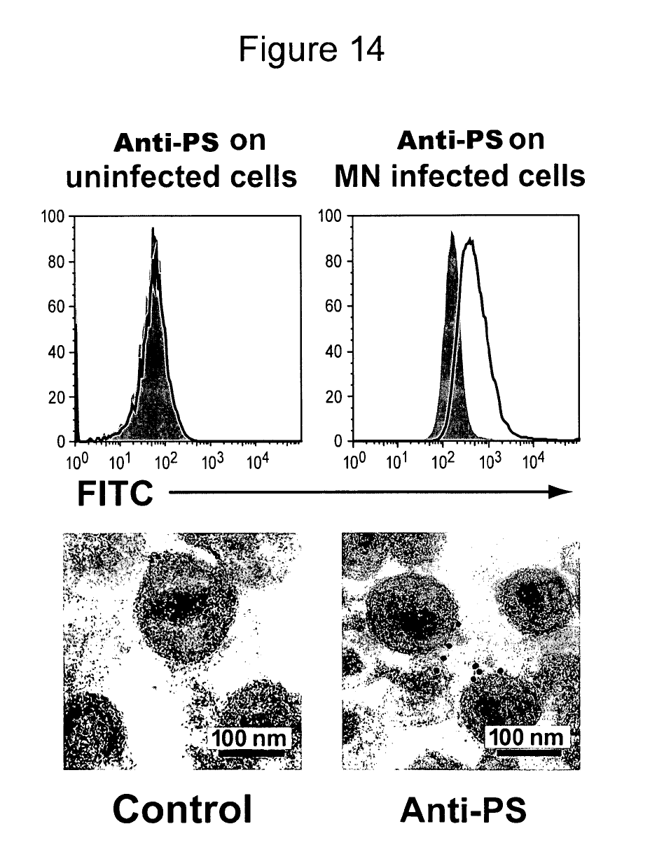

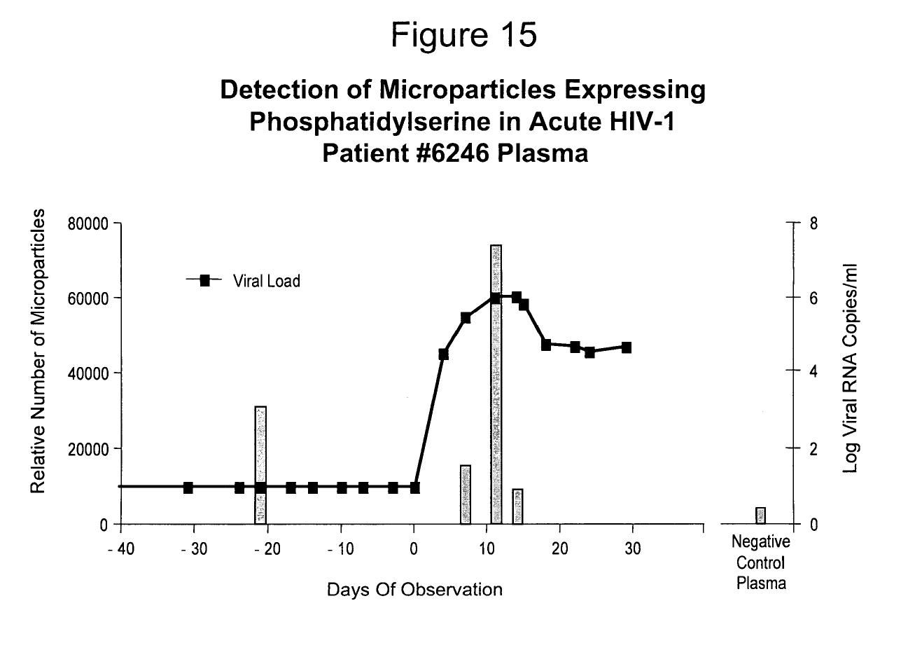

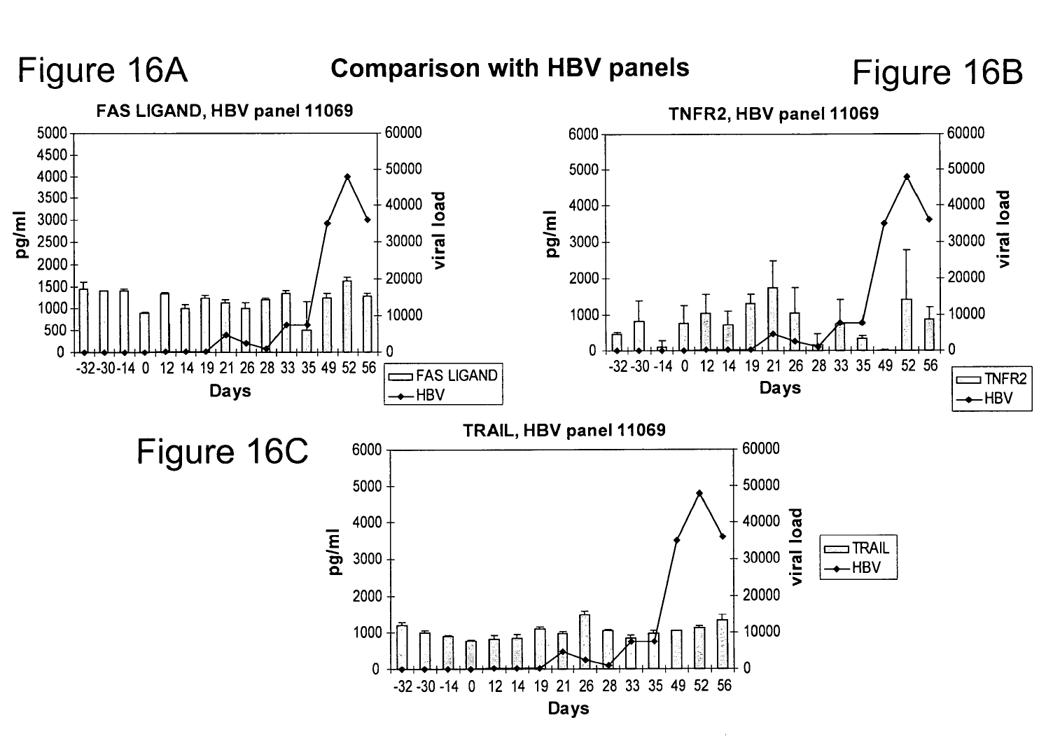

Next, flow cytometry phenotypic analysis of the microparticles in AHI sample 6246-15 was performed (FIG. 10). FIG. 11 shows the forward and side scatter of background with phosphate buffered saline, pH 7, purified microparticles from staurosporine treated Jurkat T cells, and the microparticles in patient 6246 plasma on day 11 at the time of peak viremia. FIG. 11 shows the microparticles in plasma with each panel counted for 2 minutes. FIG. 12 shows the phenotype of either purified Jurkat microparticles (pMP) or patient 6246 MP and shows that the microparticles can be phenotypically analyzed and that the AHI patient microparticles are 78.6% CD3+, 53% CD45+. CD45 is a surface molecule of T and monocyte cells that is not incorporated into HIV virions (Esser et al, J. Virol. 75:6173-6182 (2001)). Thus, about one half of the microparticles are likely virions in FIG. 12 and one half of the particles are apoptotic particles from HIV-uninfected cells or do not contain HIV genetic material. Of importance, most of the microparticles from patient 6246 are phosphatidylserine (PS) positive (FIG. 13). Similarly, HIV-infected cells are PS+, as shown in FIG. 14.

### Example 2

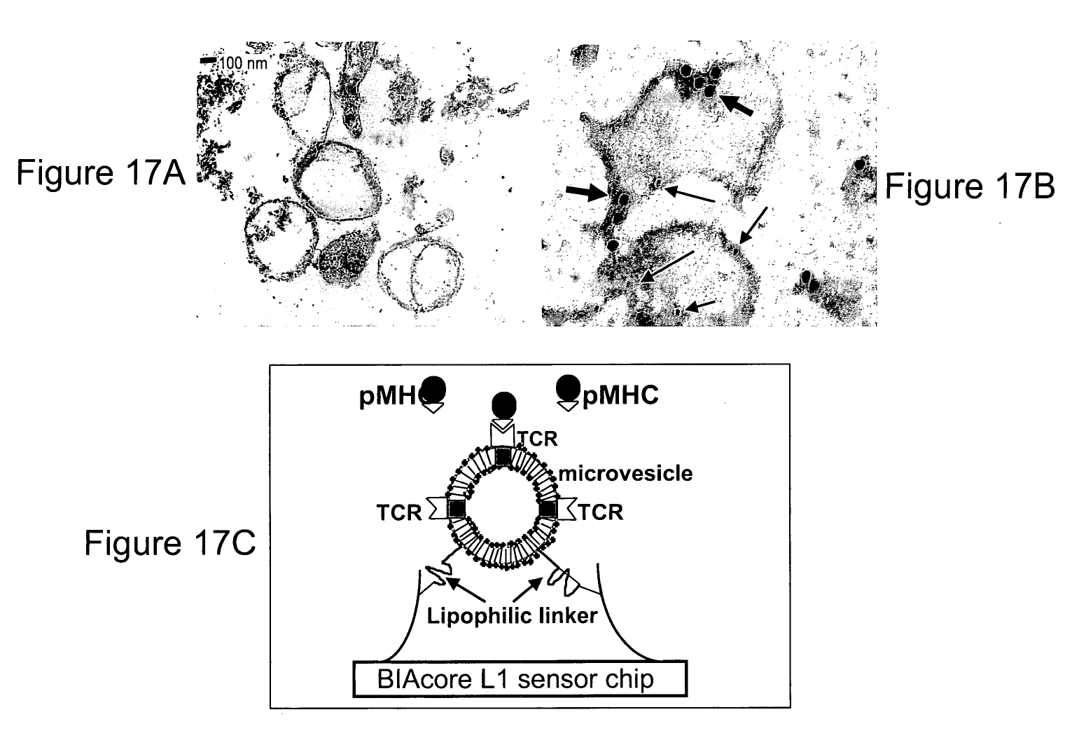

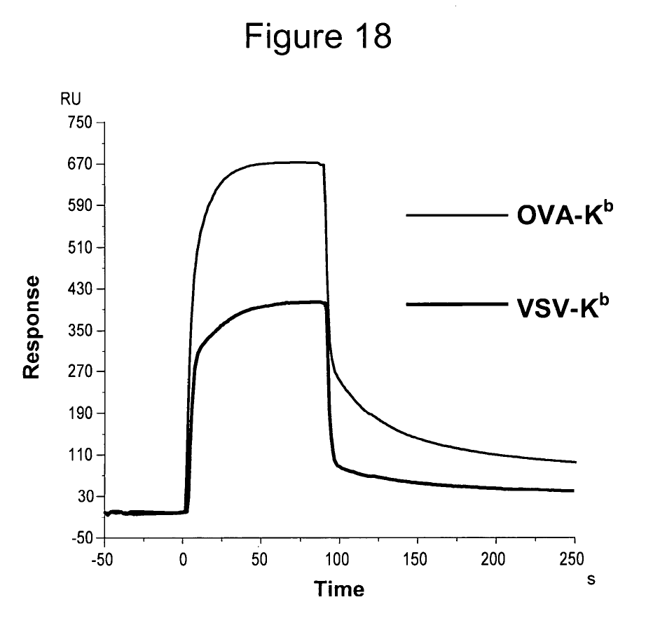

A Surface Plasmon Resonance (SPR) based proteomics assay has been developed for characterization of T cell derived microparticles. The assay allows characterization of micorparticles released from immune cells. FIGS. 17 and 18 illustrate the application of the SPR assay for the characterization of T cell microparticles. The developed assay described here can be used to monitor the status of antigen-specific T cell responses in terms of TCR specificity, protein phosphorylation and functional markers of activation and apoptosis.

### Characterization of Antigen Specific T Cell Microparticles Using MHC Class I and MHC Class II Tetramers.

Microparticles are released from immune cells like T or B cells or antigen presenting cells upon activation or apopotosis (Distler et al, Athritis & Rheumatism 52:3337-3348 (2005)). However, these microparticles differ quantitatively and qualitatively and vary depending upon the inducing stimulus (Jimenez et al, Thromb. Res. 109:175-180 (2003); Distler et al, Proc. Natl. Acad. Sci. 102:2892-2897 (2005); Kolowas et al, Scand. J. Immunol., 61:226-233 (2005)). Since these particles carry membrane derived from the parental cell, characterization of surface antigens can be used to identify the source of the microparticles. Microparticles have been reported to contain surface proteins of the immunoglobulin family (TCR, BCR), glycosylphosphatidylinositol-(GPI—) anchored molecules and members of the tetraspan family (Denzer et al, J. Cell Sci. 113:3336-3374 (2000); Koopman et al, Blood 84:1415-1420 (1994); Heijnen et al, 94:3791-3799 (1999)).

The SPR assay described here is a highly sensitive assay for use in detecting antigen specific T cells using peptide-MHC tetramers. In the study described here, T cell microvesicles were isolated from murine OT1 T cells, which express Vα2Vβ5 TCR specific for SIINFEKL-K^{b} (Ova-K^{b}) complex. These microvesicles are heterogenous in size and as assessed by dynamic light scattering and transmission electron microscopy their hydrodynamic radius vary form 400 nm to 70 nm (FIG. 17A). Microvesicles have been characterized that are spontaneously released in culture, upon cell lysis and following sucrose density centrifugation to harvest detergent resistant microdomains (DRM). The DRM have been reported to be enriched in TCR/CD3 complex and co-receptors (CD8/CD4) molecules (Montixi et al, The EMBO J. 17:5334-5348 (1998)).

Reported here is the development of a SPR based assay in which the microvesicles were first anchored on a lipid linker immobilized on the surface of L1 sensor chip (BIAcore Inc.) (FIG. 171C). About 1600 Response Unit (RU) of OT1 microvesicles were anchored on the L1 sensor chip and, following a brief period of stabilization of the surface, BSA (0.5 mg/mL) was injected for 5 min to block non-specific binding. Injection of Ova-K^{b} complex gave specific binding to the anchored OT1 microvesicles when compared to the control, VSV-K^{b} complex (FIG. 18). This demonstrates that the SPR assay can be used to monitor the presence of microparticles released from T cells upon activation or apoptosis. Additionally, the capture of microvesicles on the L1 sensor surface in RU units can be quantitated using standard synthetic microvesicles of known size and total phospholipid content. Thus, it is possible to monitor the release of microvesicles from T cells in samples from HIV infected individual and determine the antigen specificity of the TCR on the T cells undergoing apopotosis. The T cell microvesicles will be selectively captured on anti-CD3 immobilized surface and then the antigen specificity of the captured microparticles will be determined using MHC class I or MHC class II tetramers.

The assay described above is not restricted to T cell microparticles but includes characterization of microparticles from B cells as well. B cell specific tetramers have been developed by (see U.S. Prov. Appln. 60/840,423) and have been tested for specificity in SPR binding assays and by flow cytometry.

### Characterization of Protein Phosphorylation Status of T Cell Microparticles.

Microparticles consist of shed plasma membrane fragments and include cytoplasmic elements (Distler et al, Athritis & Rheumatism 52:3337-3348 (2005)). For T cell microparticles, Src kinases, Lck and Fyn, and the adapter protein LAT are associated with lipid microdomains and are the key components involved in T cell signaling (Zhang et al, Immunity, 9:239-246 (1998); Resh et al, Nature, 387:617-620 (1997); Schade & Levine, Biochem. Biophys. Res. Commun., 296:637-643 (2002)). Thus the methodologies described will serve to study the correlation of protein phosphorylation status identified in microparticles with immune T cell activation or HIV infection.

The strategies employed here include first the characterization of T cell microparticles in terms of antigen specificity using MHC tetramers as described above, and then to define the phoshorylation status of the identified antigen specific microparticles. The captured microparticles will be eluted from the BIAcore sensor surface using the BIAcore 300 recovery capability. The eluted particles will then be lysed and then the phosphorylation status determined by: a) immunoblotting with anti-signaling molecule antibodies (Src kinases, Lck, Fyn, LAT); and b) 2D-liquid chromatography followed by identification by mass spectrometry. Eluted material will be separated using 2D-LC and individual phosphorylated proteins will be analyzed using a MALDI-TOF/TOF. Proteins identified using this approach will be verified to be phosphorylated using conventional immunoblotting with anti-phospho antibodies. This application allows characterization of released microparticles in order to identify the activation state and signaling pathways in immune cells and their changes upon exposure to HIV infection.

### Characterization of Microparticles to Determine the Functional Status of the Parental Cell.

As with HIV-1 viruses that incorporate cell specific membrane proteins into its viral envelope in the process of budding from infected cells (Hioe et al, J. Virol. 75:1077-1088 (2001); Laio et al, AIDS Res. Hum. Retro. 16:355-366 (2000)), budding microparticles carry on their cell surface protein markers derived from the parental cell. These surface markers are tell-tale sign of the functional status of the T cells from which they came from. In addition to flow cytometric based phenotyping of microparticles, the capture of microparticles from T cells using the SPR assay described above is a novel methodology for determining the activation status of the T cells. The functional status of the T cells is determined by monitoring the expression of activation markers (CD69, CD25), apopotosis markers (PD1, TRAIL receptors, FAS, Fas L).

### Example 3

A critical event in HIV-1 and SIV infection is virus-induced massive CD4+, CCR5+ T cell loss that is severe in gut associated lymphoid tissues (GALT) (Guadalupe et al, J. Virol. 77:11708-11717 (2003), Brenchley et al, J. Exp. Med. 200:749-759 (2004), Mehandru et al, J. Exp. Med. 200:761-770 (2004)). Depletion of GALT CD4 T cells has been documented at peak viral load in acute SIVmac239 infection (Veazey et al, Science 280:427-431 (1998), Haase, Nat. Rev. Immunol. 5:783-792 (2005), Li et al, Nature 434:1148-1152 (2005), Mattapallil et al, Nature 434:1093-1097 (2005)), as well as within weeks of HIV-1 transmission in man (Guadalupe et al, J. Virol. 77:11708-11717 (2003), Brenchley et al, J. Exp. Med. 200:749-759 (2004), Mehandru et al, J. Exp. Med. 200:761-770 (2004)). During acute SIV infection, 30-40% of memory CD4+ T cells are infected (Veazey et al, Science 280:427-431 (1998), Haase, Nat. Rev. Immunol. 5:783-792 (2005), Li et al, Nature 434:1148-1152 (2005), Mattapallil et al, Nature 434:1093-1097 (2005)). The mechanisms of immune cell death during acute HIV-1 infection are not known, but may involve induction of apoptotic pathways by HIV Tat, Vpr or gp120 proteins (Badley et al, Blood 96:2951-1964 (2000), Chase et al, Trends Pharmacol. Sci. 27:4-7 (2006), Boya et al, Biochim. Biophys. Acta 1659:178-189 (2004)), HIV-1 infection of CD4+ T cells (Guadalupe et al, J. Virol. 77:11708-11717 (2003), Mehandru et al, J. Exp. Med. 200:761-770 (2004), Veazey et al, Science 280:427-431 (1998), Li et al, Nature 434:1148-1152 (2005)), and induction of uninfected cell death due to killing by molecules such as tumor necrosis factor related apoptotis inducing ligand (TRAIL) (Lum et al, J. Virol. 75:11128-11136 (2001), Herbeuval et al, Clin. Immunol. 123:121-128 (2007)).

The time from HIV-1 transmission to establishment of the latently infected pool of CD4 T cells has been termed the window of opportunity within which a preventive HIV-1 vaccine has to extinguish HIV-1 (Johnston et al, N. Engl. J. Med. 356:2073-2081 (2007, Wong et al, Biology of Early Infection and Impact on Vaccine Design, pgs. 17-22 (Caister Academic Press, Norfolk, UK (2007)). The latent pool is established at least by the time of symptomatic acute HIV-1 infection at the time of seroconversion (−25 days after transmission), although the exact earliest time of establishment of the latent CD4 T cell pool is not known (Wong et al, Biology of Early Infection and Impact on Vaccine Design, pgs. 17-22 (Caister Academic Press, Norfolk, UK (2007), Chun et al, Proc. Natl. Acad. Sci USA 95:8869-8873 (1998)). Adaptive CD4, CD8 and B cell antibody responses to HIV-1 do not appear during the eclipse or viral load ramp-up phases of HIV-1 infection, but rather appear coincident with the fall in viral load (VL) and appearance of acute infection symptoms at the end of the window of opportunity (Reynolds et al, J. Virol. 79:9228-9235 (2005), Abel et al, J. Virol. 79:12164-12172 (2005), Fiebig et al, AIDS17:1871-1879 (2003)). Thus, study of the events that transpire from transmission until the onset of plasma viremia (the eclipse phase) and during the viral load ramp-up phase of acute HIV-1 infection are critical to understanding why immune responses do not occur earlier after HIV-1 transmission, and to define what a successful vaccine must overcome to extinguish HIV-1.

In the study described below, the hypothesis is raised that, in addition to gut CD4 T cell loss, delay in HIV-1 protective immune responses early on after HIV-1 transmission may involve the production of elevated levels of immunosuppressive moieties such as TRAIL, TNFR2 and Fas ligand as well as plasma microparticles. If elevations in immunosuppressive molecules, coupled with early CD4+ T cell death, occur early on after HIV-1 transmission, then this would define a protected time for HIV-1 to replicate while anti-HIV-1 T or B cell responses were suppressed.

To study the eclipse and early viral load ramp-up phases of acute HIV-1 infection, archived plasma of plasma donors with samples available before, during, and after HIV-1 viral load ramp-up have been used (Fiebig et al (AIDS 17:1871-1879 (2003)). An initial burst of soluble TRAIL was found in plasma soon after the appearance of HIV-1 in plasma, corresponding to ˜17 days following transmission. Also observed were later elevated plasma TNFR2, Fas ligand and plasma microparticles (MP) levels around the peak of plasma VL. These data implicate TRAIL as an early mediator of cell death in acute HIV-1 infection, and demonstrate a narrow window of opportunity in which a HIV-1 vaccine must extinguish the transmitted virus.

### EXPERIMENTAL DETAILS

Plasma Samples. Seroconversion panels (HIV-1+/HCV−/HBV−, n=30, HIV-1−/HCV−/HBV+, n=10, and HIV-1−, HCV+/HCV−, n=10) were obtained from ZeptoMetrix Corporation (Buffalo, N.Y.). Each panel consisted of sequential aliquots of plasma (range 4-30) collected approximately every 3 days during the time of acute infection with HIV-1 (Huang et al, J. Immunol. 177:2304-1313 (2006)). HIV-1−/HCV−/HBV-human plasmas (n=25) were obtained from Innovative Research (Southfield, Mich.). All studies were approved by the Duke University human subjects institutional review board.

Viral Load Testing. Viral load testing of HIV-1 plasma plasma donor panels was performed by Quest Diagnostics (Lyndhurst, N.J.) (HIV-1 RNA PCR Ultra). HCV and HBV viral loads were preformed by Zeptometrix; select HCV viral loads were provided by Philip Norris, Blood Systems Research Institute, San Francisco, Calif.

ELISAs For Plasma Markers of Apoptosis. ELISAs for Fas, Fas Ligand, TRAIL, (Diaclone, Besancon Cedex, France), and TNFR2 (Hycult Biotechnology, Uden, The Netherlands) were performed according to the manufacturer's directions. Plasma was assayed undiluted (TRAIL), diluted 1:10 (TNFR2) or diluted 1:2 (Fas Ligand). Increases in plasma analytes over time were defined as >20% increase of values after TO versus before TO.

Apoptotic Microparticle (MP) Quantification. The number of MP in each plasma sample was determined with flow cytometry. All flow cytometry analyses were performed on the LSRII Flow Cytometer (BD Biosciences, San Jose, Calif.), and data analyses were performed using FlowJo software (Ashland, Oreg.). All buffers (PBS without calcium and magnesium (Cellgro, Herndon, Va.) and formaldehyde (Sigma, St. Louis, Mo.)) were filtered with a 0.22 μm filter (Millipore, Billerica, Mass.), before use in any MP experiment. The buffer used to dilute plasma samples (1% Formaldehyde in PBS without calcium and magnesium), was used to define the background MP count (−150 events counted in 60 seconds on the flow cytometer). To define the MP gate, FluoSpheres Fluorescent Microspheres (Molecular Probes, Eugene, Oreg.), ranging in size from 0.1 μm to 1 μm, were analyzed on the flow cytometer. The MP gate was drawn around the beads, encompassing the 0.1 μm, 0.2 μm, 0.5 μm, and 1.0 μm beads. Each plasma sample was diluted 1:100 and 1:1000 in 1% formaldehyde/PBS, and data acquired for 60 seconds. Optimal sample dilutions were determined experimentally, with the acceptance criteria being the dilution of plasma with abort counts<5%, and noise to signal ratios<0.1 (noise to signal ratio=background MP count in PBS/experimental plasma MP count).

Microparticle Phenotypic Analysis. MP were analyzed by flow cytometry for cell surface markers as described (Hosaka et al, J. Infect. Dis. 178:1030-1039 (1998), Stacey et al and the NIAID Centre for HIV/AIDS Vaccine Immunology. Elevations in plasma levels of innate cytokines prior to the peak in plasma viremia in acute HIV-1 infection (2007), Clark et al, N. Engl. J. Med. 324:954-960 (1991)). Plasma samples (1-2 ml) were diluted in 5 ml of filtered saline, and then filtered through a 5 μM filter (Pall Corporation, East Hills, N.Y.). The diluted samples were then centrifuged (1 hr at 200,000×g at 4° C.) (Sorvall RC M150 GX, Thermo Fisher Scientific, Waltham, Mass.). The top 2.5 ml of supernatant was removed, 2.5 ml of fresh saline added, and samples were centrifuged again (1 hr at 200,000×g at 4° C.). The pellet was washed ×2 in 1 ml of filtered saline; after the last wash, 900 μl of the supernatant was removed, and the pellet resuspended in the remaining 200 μl of saline. Ten μl of MP suspension was incubated with an antibody and/or annexin V (total volume of 100 μl×20 minutes, 20° C., in the dark). Saline with 1% BSA (Sigma) was used as staining buffer for incubation with antibodies, and 2.5 mM CaCl₂ added to the buffer for annexin V staining. For annexin V controls, 50 mM EDTA was added to the buffer. After incubation, the volume was adjusted to 500 μl with saline/formaldehyde, and analyzed by flow cytometry within 24 hours. Conjugated antibodies included mouse anti-human CD45-PE, CD3-PE, CD61-PerCp, CCR5-PE, and isotype controls (BD Biosciences, San Jose, Calif.) and annexin V conjugated to AlexaFluor 647 (Molecular Probes, Eugene, Oreg.).

Electron Microscopy of Plasma Microparticles. Eight ml of plasma was diluted 1:5 in filtered saline and MP were pelleted (200,000×g×1 hr, 4° C.). Pellets were washed ×2 (100,000×g×30 min.) in 1 ml of saline. The MP pellet was resuspended in 500 μl of saline and overlaid onto 1 ml of a 40% sucrose solution (in saline) and MP were centrifuged (100,000×g×90 min.). The pellets were fixed (1% formaldehyde, 4° C. overnight), pelleted (100,00×g×60 min.), then fixed in 1% osmium tetroxide. Ultrathin sections were cut and post stained with uranyl acetate and examined on a Philips CM12 electron microscope (FEI Co., Hillsboro, Oreg.).

In Vitro Mucosal B cell Culture Model. Tonsils were obtained from pediatric and adult patients who underwent tonsillectomy at the Duke University Medical Center. Tonsils were placed in transport media (RPMI 1640 with L-Glutamine (Gibco, Carlsbad, Calif.), supplemented with 200 U/ml penicillin G, 200 μg/ml streptomycin, 50 μg/ml gentamicin, and 1 μg/ml amphotericin B (Sigma) upon excision and transported to the laboratory for processing within 4 hours. The tonsils were washed extensively in RPMI 1640 with L-Glutamine supplemented with 100 U/ml penicillin G, 100 μg/ml streptomycin, 50 μg/ml gentamicin, and 2 μg/ml amphotericin B to prevent bacterial and fungal contamination. To isolate tonsillar lymphocytes, the specimens were mechanically minced and teased with sterile forceps, and the resulting single-cell suspension was processed through a 70 μm nylon cell strainer (BD Biosciences). Lymphocytes were separated with lymphocyte separation media (Fisher Scientific, Pittsburg, Pa.) and washed twice. Cell number and viability were determined with a Guava EasyCyte Mini (Hayward, Calif.) per the manufacturer's instructions. Cells were cultured in RPMI 1640 supplemented withl 00 U/ml penicillin G, 100 μg/ml streptomycin, 25 μg/ml gentamicin, 1 μg/ml amphotericin B, and 10% FBS (Gemini Bioproducts, West Sacramento, Calif.), at a density of 1×10⁶ cells/ml in total volumes of 1 ml in polystyrene 5 ml round-bottom tubes (BD Biosciences). To stimulate antibody production, cells were cultured with an optimized cocktail of oCpG (6 μg/ml) (Coley Pharmaceutical Group, Wellesley, Mass.), and pokeweed mitogen extract (PWM) 1/2000 (Crotty et al, J. Immunol. Methods 286:111-122 (2004)). After 5 days of culture, supernatants were harvested and total IgG and IgA present in culture supernatants was quantified by ELISA. Briefly, 96-well ELISA plates (Corning, Corning, N.Y.) were coated with purified murine anti-human IgG Fc (HRL, Baltimore, Md.) or purified murine anti-human IgA (BD Pharmingen) in 0.1 M NaHCO₃ overnight. Wells were washed x3 with wash buffer (PBS with 0.1% Tween (Sigma)). Wells were blocked with dilution buffer (1% FBS, 0.5% BSA (Sigma) and 0.05% Tween) for 2 hours at 20° C. Wells were washed x3, and supernatants were added diluted 1:2 with dilution buffer. Plates were incubated overnight at 4° C., and washed ×3. Biotinylated murine anti-human IgG Fc (HRL) or biotinylated murine anti-human IgA (BD Pharmingen) was added, and plates were incubated for 2 hours at 20° C. After washing ×3, horseradish peroxidase streptavidin (Vector Laboratories, Burlingame, Calif.) was added and plates incubated for 45 minutes at 20° C. Plates were washed ×3, and the assay was developed using a 3,3′,5,5′ tetra-methylbenzidine (TMB), substrate and stop solution system (KPL, Gaithersburg, M.D.). Standard curves were constructed using serial dilutions of purified human IgG and IgA (Sigma).

Microparticles were generated by treatment of normal donor PBMC or tonsil cells with staurosporine Bell et al, Am. J. Physiol. Cell Physiol. 291:C1318-C1325 (2006)). Cell viability and cell counts were performed on the Guava EasyCyte Mini, and 5×10⁷ cells were cultured in 5 ml RPMI 1640 with L-glutamine supplemented with 25 μg/ml of gentamicin, 10% FBS, and 1 μM staurosporine (Sigma). After culture overnight, the cells and supernatant were collected, and centrifuged ×2 (5 min at 400×g at 4° C.). The cell-free supernatant was then centrifuged in an ultracentrifuge to harvest the MP (30 min at 200,000×g at 4° C.) and MP were washed ×1 with RPMI 1640, resuspended in 1000 μl of fresh RPMI, and 100 μl of MP suspension was added to select tonsil cell cultures (or varying volumes to determine dose dependency).

Statistical Analyses. Statistical Analysis was performed as outlined in the figures and descriptions thereof using boxplot analyses, Student's t tests, and Wilcoxon Rank Sum analyses. To establish a reference point throughout all the plasma seroconversion panels, Time 0 (TO) was defined as the date when viral load reached 100 copies/ml for HIV-1,600 copies/ml for HCV, and 700 copies/ml for HBV. To determine the percent increase in plasma markers of apoptosis during HIV-1, HBV, and HCV infections, the mean TRAIL, TNFR2, or Fas Ligand level before Day 0 was compared to the mean level after Day 0, and percent increase was calculated ([(mean after day 0−mean before day 0)/mean after day 0]×100).

### Results

TRAIL, TNFR2 and Fas Ligand Plasma Levels are Elevated During Acute HIV-1 Infection.

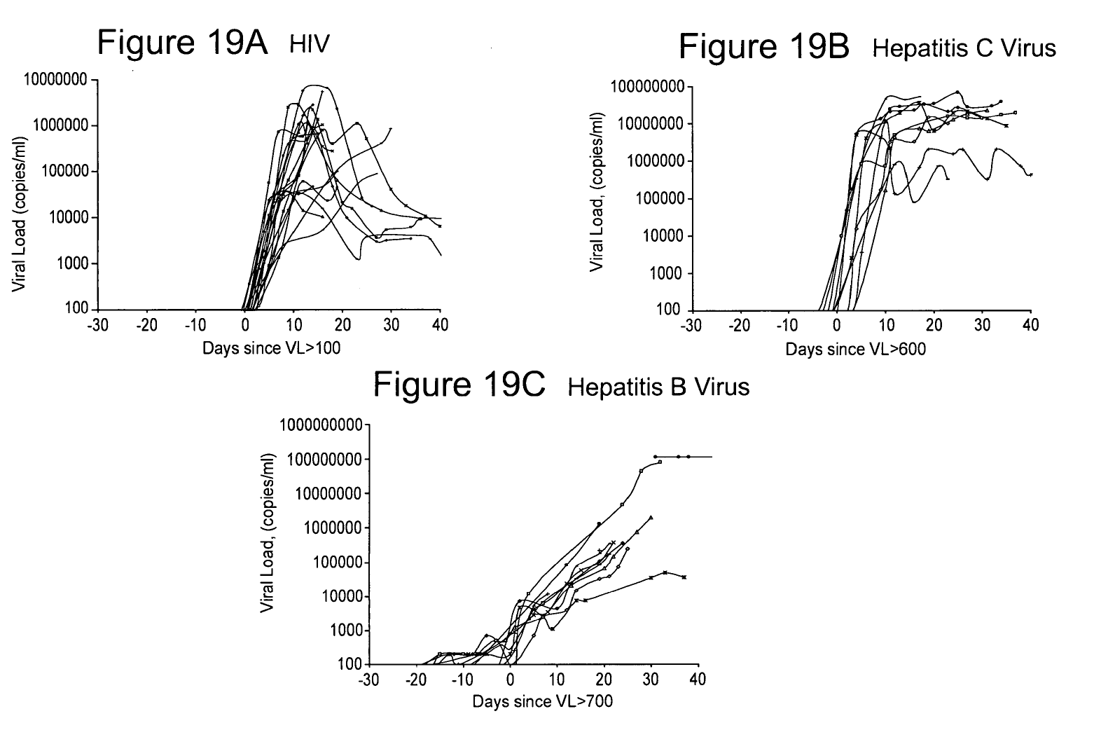

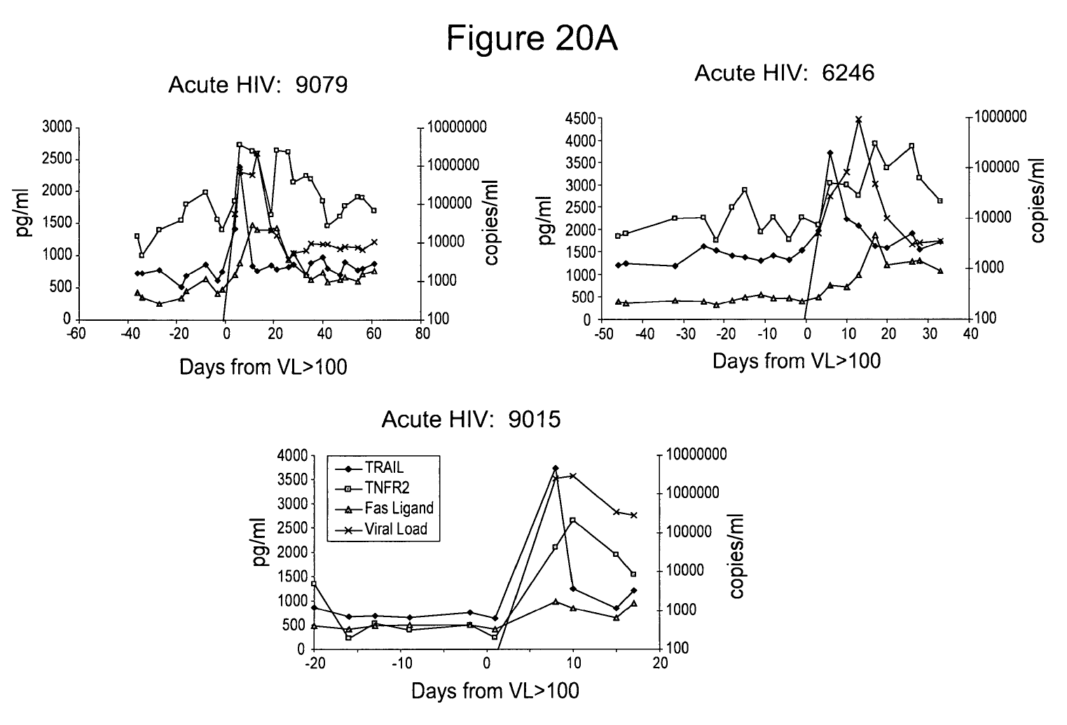

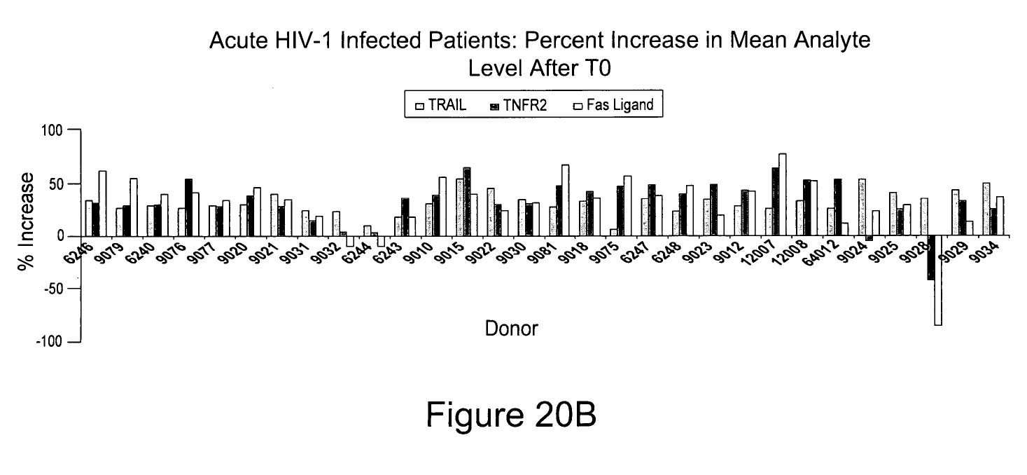

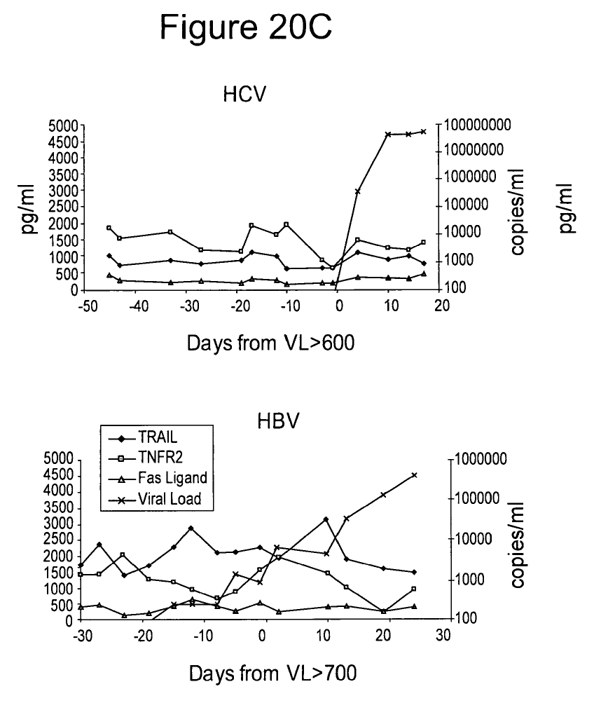

A timepoint (T0) was determined for each of 30 HIV-1, 10 HCV and 10 HBV patients, defined as the lower limits of detection for each viral load determination (FIG. 19). Soluble TRAIL, TNFR2, and Fas Ligand were next assayed in sequential plasma samples of each plasma donor (FIG. 20A). The percent change in plasma TRAIL, TNFR2 and Fas ligand levels were determined by comparing the mean analyte level before T₀ to the mean level after T₀; 27/30 demonstrated increases in TRAIL, 26/30 had increased TNFR2, and 22/30 had increased Fas ligand levels by these criteria (FIG. 20B).

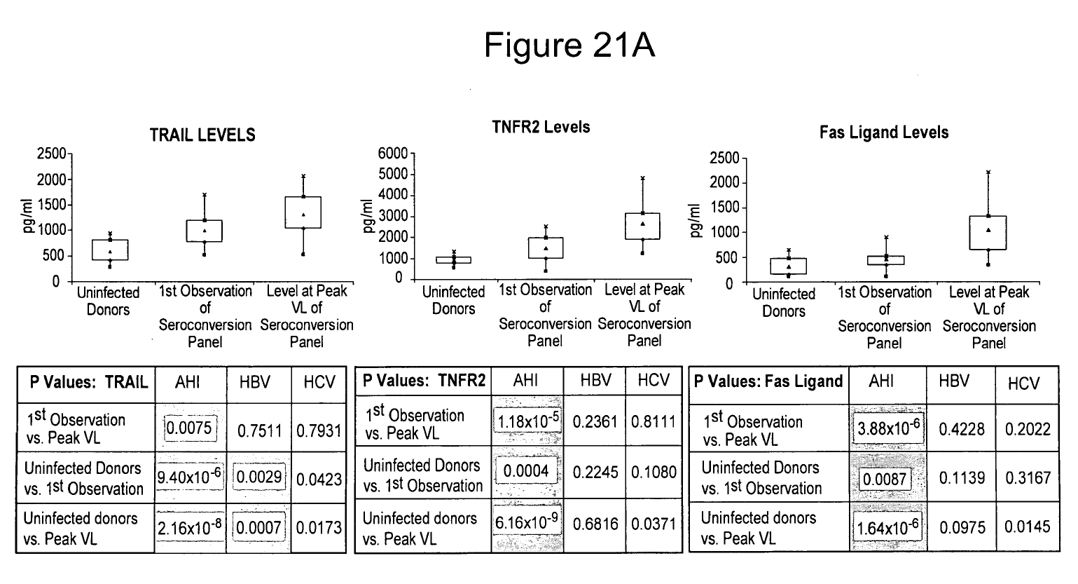

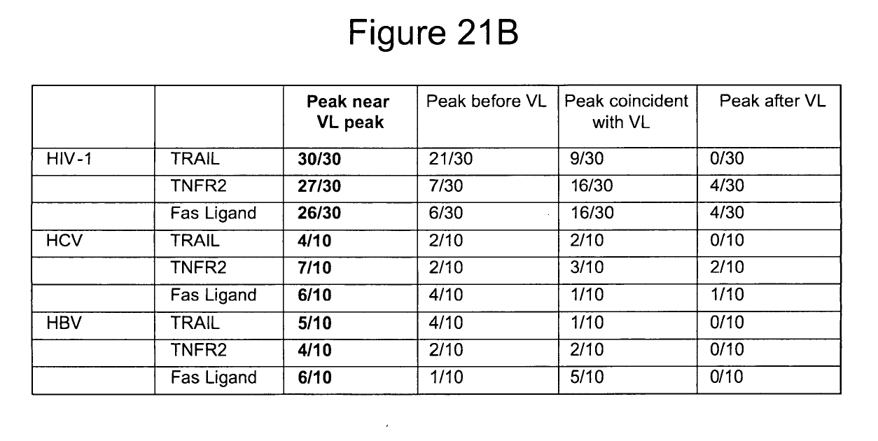

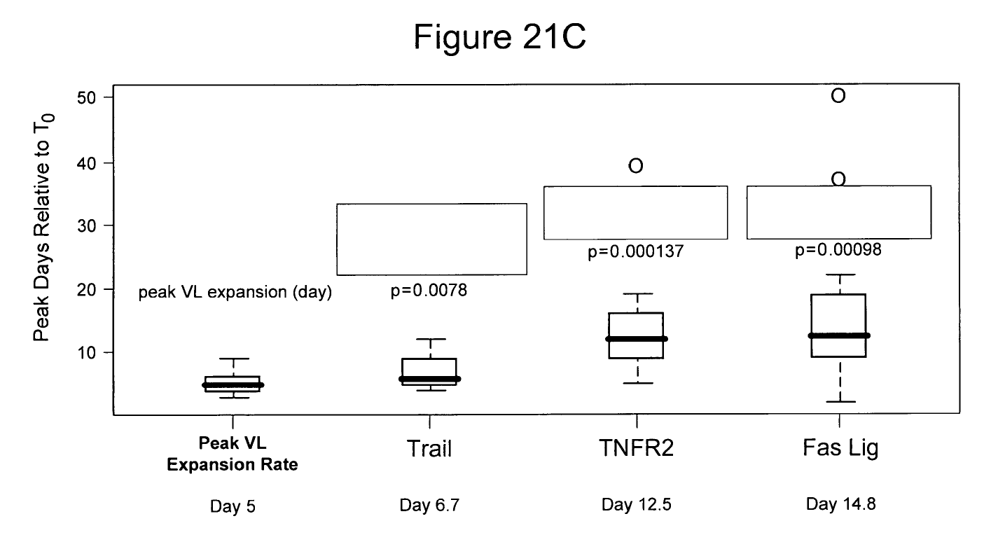

Hepatitis B and C infections have been reported to induce cell death of hepatocytes (Chase et al, Trends Pharmacol. Sci. 27:4-7 (2006), Chou et al, J. Immunol. 174:2160-2166 (2005)). As controls, HCV and HBV acutely infected subjects demonstrated a >20% rise in TRAIL, TNFR2 or Fas ligand only in 0/10, 3/10, and 2/10 HBV acutely infected subjects, and in 1/10, 6/10 and 7/10 HCV acutely infected subjects, respectively (FIGS. 20C, 21B).

Second, cell death plasma analyte levels at the time of peak viral load were compared to samples drawn from subjects before viral load ramp-up, and as well, compared with uninfected plasmas. The mean TRAIL, TNFR2, and Fas ligand levels at the time of peak viral load were significantly different from the earliest plasma sample drawn from each acute HIV-1 infected patient before T₀ (p=0.0075 for TRAIL, p=1.18×10⁻⁵ for TNRF2 and p=3.88×10⁻⁶ for Fas ligand) (FIG. 21A). The peak TRAIL, TNFR2 and Fas ligand levels were also significantly different from the levels of TRAIL, TNFR2, and Fas ligand in uninfected plasma sample controls (p=2.16×10⁻⁸, p=6.16×10⁻⁹, and p=1.64×10⁶, respectively) (FIG. 21A).

Third, to investigate the timing of peak levels of TRAIL, TNFR2 and Fas ligand compared to peak plasma VL, a determination was made of the temporal relationship between an apoptotic analyte peak compared to the peak plasma VL. Also determined was the number of subjects that had peaks in plasma cell death analytes occurring before, coincident with or following the peak in HIV-1 viral load (FIG. 21B). The majority of acute HIV-1 infection subjects (30/30 for TRAIL, 27/30 for TNFR2, and 26/30 for Fas ligand) demonstrated peak analyte levels occurring within a 30-day time frame (i.e., 15 days before, at the time of, or within 15 days after the viral load peak). Of particular interest, the majority of subjects' TRAIL levels (21/30) peaked before the peak viral load, while TNFR2 and Fas ligand levels more often peaked coincident with viral load (FIG. 21B).

Fourth, to analyze the timing of peak analyte levels relative to the rate of viral expansion during viral load ramp-up, paired Wilcoxon rank tests were performed (FIG. 21C). The day of the peak viral expansion rate indicates the day following T₀ on which the virus was replicating at the maximum rate. In 24 plasma donors in which the rate of VL expansion could be calculated, the peak viral expansion rate occurred on mean day 5.5 following T₀ (FIG. 21C). Plasma TRAIL levels peaked 1.7 days after maximal viral expansion rate (day 7.2 after T₀) while TNFR2 levels peaked 7.5 days (day 13 after T₀), and Fas ligand levels peaked 9.8 days (day 15.3 after T₀) after the time of maximal rate of viral expansion. Plasma donor HIV-1 VL reached its peak an average of 13.9 days after T0 (median 13 days, interquartile range 3 days), indicating that TRAIL levels peaked well before VL peaked, while TNFR2 and Fas ligand reached peak levels close to the time of highest VL levels.

The mean of the peak plasma TRAIL levels was 2011 pg/ml with a range of 886-4138 pg/ml. This level of TRAIL is well within the biologically relevant concentration range of an activity for induction apoptosis in immune cells²¹.

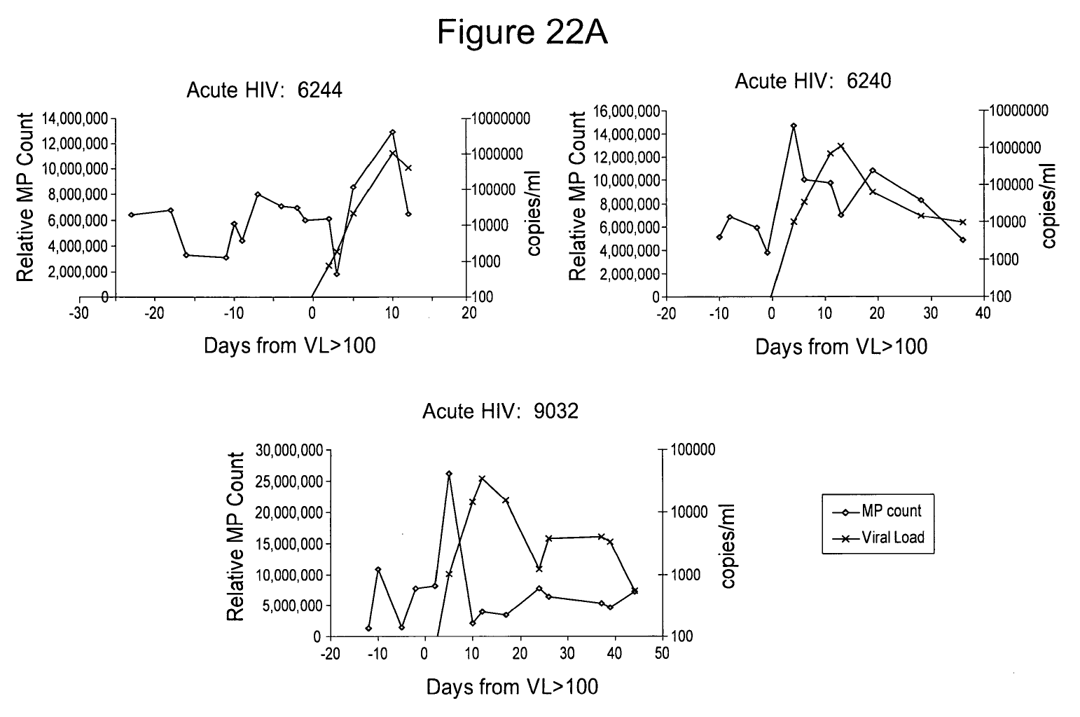

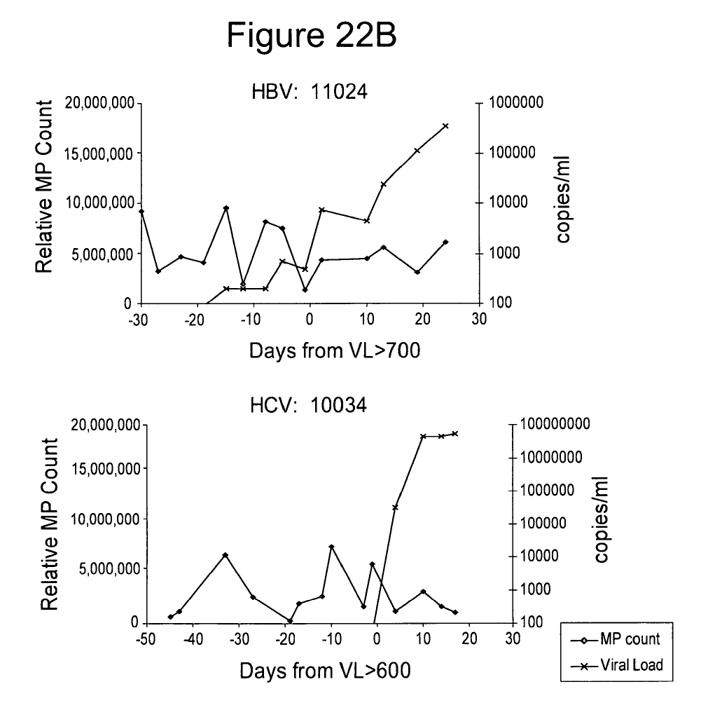

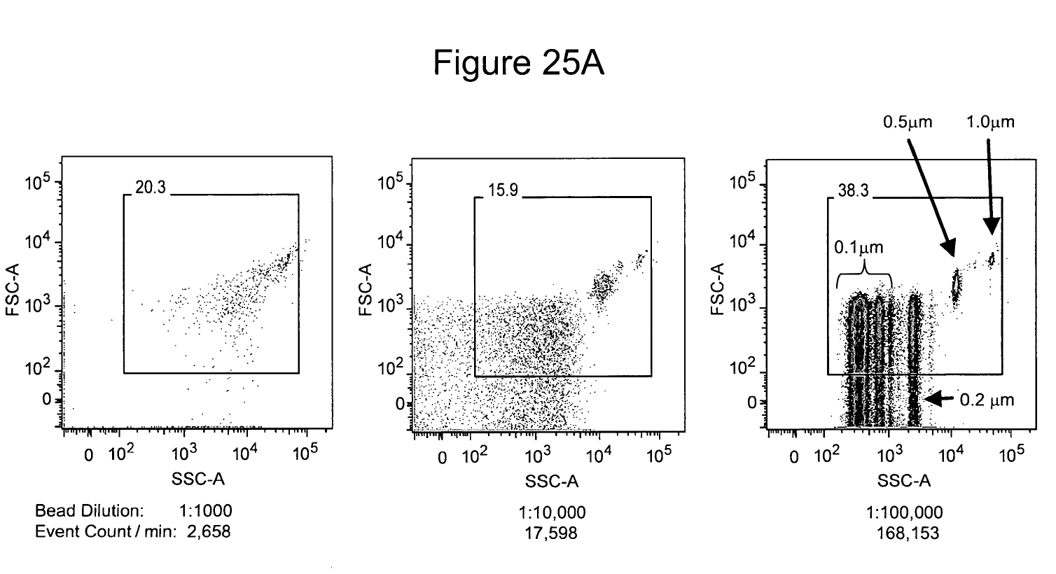

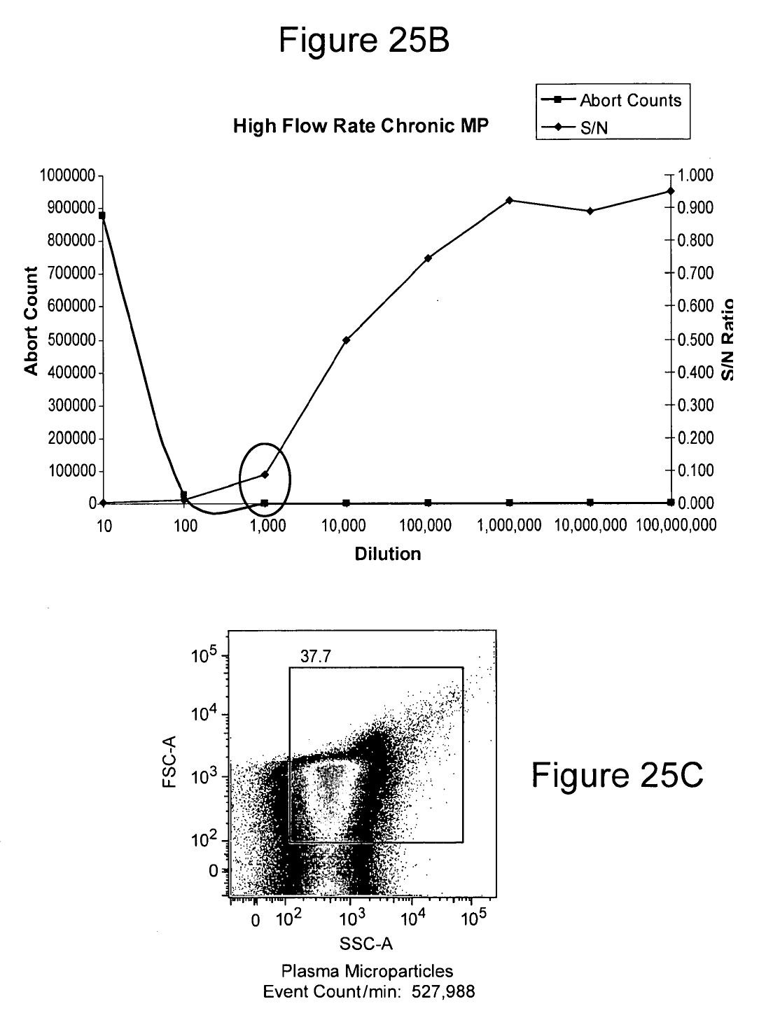

Quantitative flow cytometry analysis of plasma microparticles. Plasma MP are a normal by-product of a variety of types of activated or apoptotic cells, or are derived from multivesicular bodies (exosomes) (Distler et al, Autoimmunity 39:683-690 (2006), Piccin et al, Blood Rev. 21:157-171 (2007) (FIG. 25). Eighteen of thirty (60%) plasma donors demonstrated peak MP levels near (within 15 days before or 15 days after T₀) the peak in viral load, and 11 of these 18 peaks occurred immediately before the peak in viral load, while 4/18 peaked at the time of VL peak (Table 1 and FIG. 22A). Five of ten (50%) HCV donors had similar elevations in MP, but only two of ten (20%) HBV panels studied had elevations in MP levels near the peak VL (FIG. 22B).

TABLE 1

| | HIV | HCV | HBV |
|---|---|---|---|
| Peak MP level within | 18/30 | 5/10 | 2/10 |
| 30 days (±15 days of | | | |
| peak VL) | | | |
| Peak MP level before | 11/18 | 3/5 | 2/5 |
| peak VL | | | |
| Peak MP level | 4/18 | 1/5 | 0/5 |
| coincident with peak | | | |
| VL | | | |
| Peak MP level after | 3/18 | 1/5 | 0/5 |
| peak VL | | | |

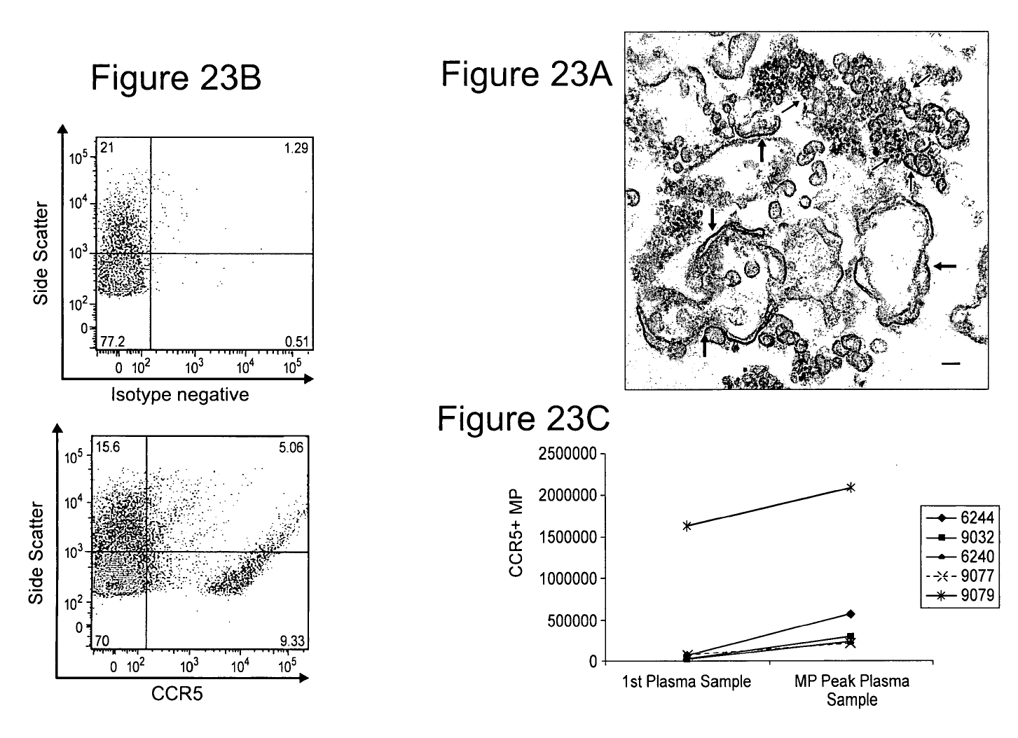

The morphology of MP at the time of peak viral load was studied from acute HIV-1 infection patient 6244 on sucrose gradient purified MP from the time of both peak VL and peak MPs (day 10 in FIG. 22A) and found to be heterogeneous in size ranging from 10 nm to 1000 nm (FIG. 23A).

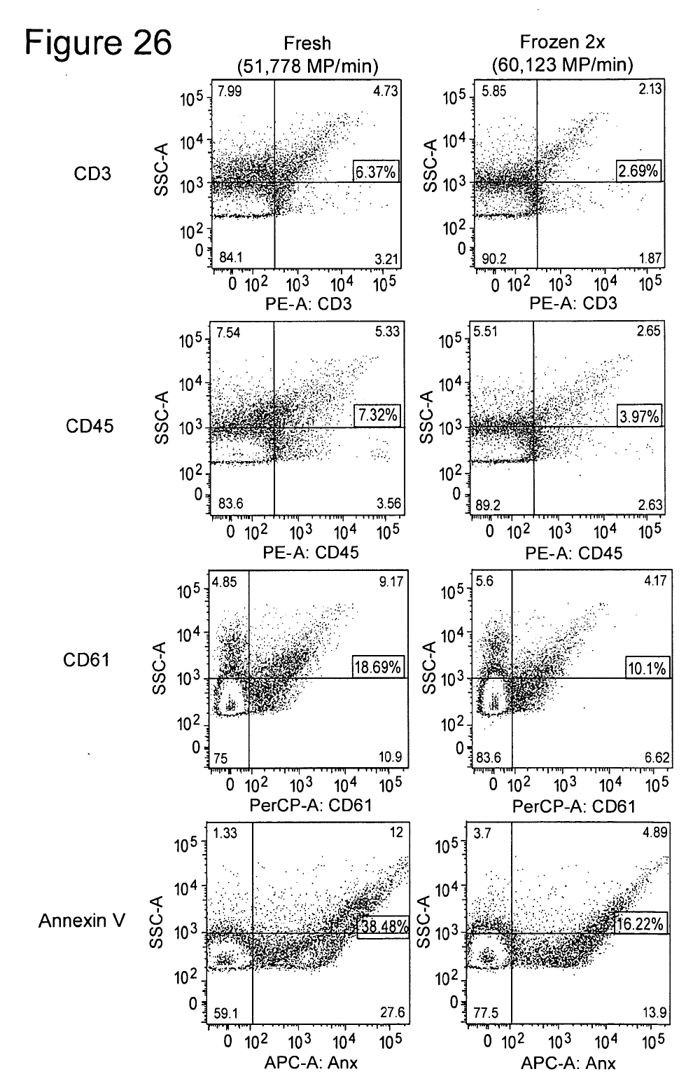

Phenotypic characterization of plasma microparticles. Most studies of phenotypic analysis of MP used fresh plasma that was processed within hours (Jy et al, J. Thromb. Haemost. 2:1842-1851 (2004)), whereas the plasma donor samples in this study have been frozen/thawed at least twice. It was found that two freeze/thaw cycles markedly decreased the percentage of CD3, CD45+, CD61+ and annexin+ MPs (FIG. 26). Thus, unable to accurately quantitate the phenotype of plasma MP, MP was analyzed for annexin and CCR5 expression in a qualitative manner to determine if MP expressing annexin or CCR5 were present at the time of peak MP in five plasma donor peak MP samples vs. uninfected first samples. Both annexin+ and CCR5+ MP were present in the plasma donor plasmas; for annexin, the mean %+ at MP peak was 12%, range 2.3%-38.0%, and for CCR5+ MP, %+mean at the MP peak was 5.7,%, range 1.1%-12.6% (FIGS. 23B, 23C). For CCR5+ but not for annexin+MP, there was a trend in higher MP numbers at the time of peak MP compared to the first panel sample (FIG. 23C).

While the average peak HIV-1 VL level was 1,421,628 copies/ml, the average peak of total MPs was 606,881,733/ml. Thus, there was an average of 427-fold more MP than virions present in plasma at their peaks during acute HIV-1 infection.

TABLE 2

| Subject | Viral Load (copies/ml) | MP (MP/min) |
|---|---|---|
| 6246 | 922,270 | 541,145,000 |
| 9079 | 2,170,000 | 828,050,000 |
| 6240 | 1,097,490 | 737,750,000 |
| 9076 | 697,960 | 3,012,125,000 |
| 9077 | 1,114,040 | 121,575,000 |
| 9020 | 45,995 | 1,916,050,000 |
| 9021 | 5,417,090 | 834,200,000 |
| 9031 | 88,594 | 1,358,700,000 |
| 9032 | 34,213 | 1,309,650,000 |
| 6244 | 1,069,090 | 647,085,000 |
| 6243 | 60,358 | 279,425,000 |
| 9010 | 31,770 | 631,800,000 |
| 9015 | 2,912,250 | 461,740,000 |
| 9022 | 1,361,290 | 220,365,000 |
| 9030 | 660,060 | 178,255,000 |
| 9081 | 90,564 | 645,080,000 |
| 9018 | 372,977 | 806,150,000 |
| 9075 | 844,020 | 1,084,000,000 |
| 6247 | 2,821,080 | 319,300,000 |
| 6248 | 5,382,888 | 602,415,000 |
| 9023 | 1,219,850 | 234,600,000 |
| 9012 | 1,032,790 | 432,750,000 |
| 12007 | 47,422 | 60,600,000 |
| 12008 | 6,486,240 | 3,081,000,000 |
| 64012 | 2,228,480 | 119,605,000 |
| 9024 | 69,105 | 352,050,000 |
| 9025 | 3,706,202 | 257,700,000 |
| 9028 | 57,186 | 524,100,000 |
| 9029 | 45,188 | 403,100,000 |
| 9034 | 562,373 | 757,700,000 |
| | mean: 1,421,628 | mean: 758,602,167 |
| | | *mean: 606,881,733 |

*Corrected MP levels - Quantitation of MP levels in fresh plasma vs. 2x frozen/thawed plasma showed a ~20% increase in MP levels in 2x frozen/thawed plasma. Thus, the mean MP level was corrected by ~20%.

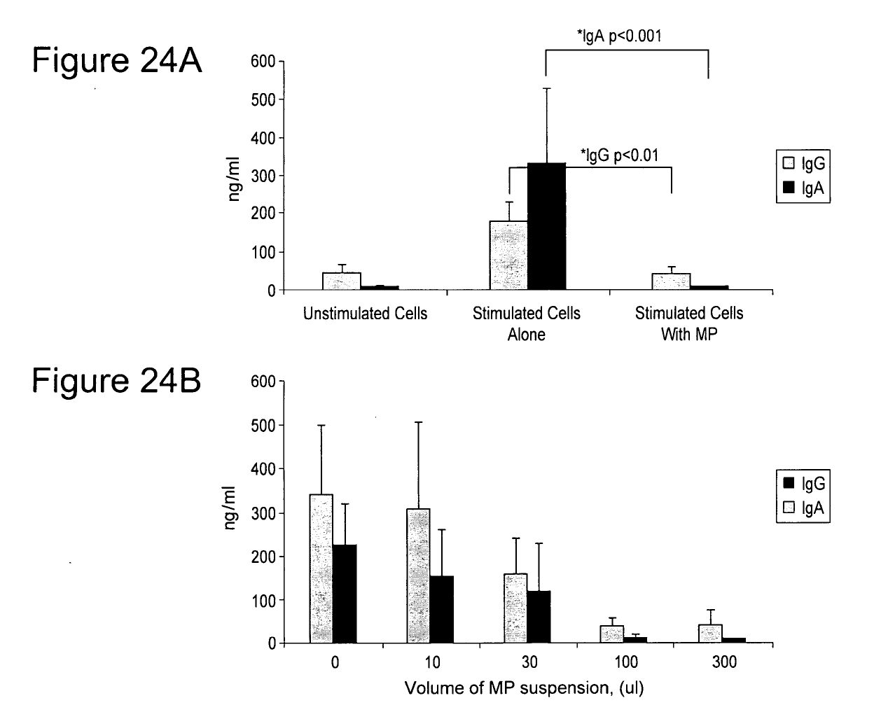

MP-Induced B Cell Suppression In Vitro. While plasma MPs have potent known suppressive effects on macrophages and DCs (Hoffmann et al, J. Immunol. 174:1393-1404 (2005), Huynh et al, J. Clin. Invest. 109:41-50 (2002)), only one study has suggested MP may inhibit B cell activation (Koppler et al, Eur. J. Immunol. 36:648-660 (2006)). There was particular interest in MP effects on human memory B cell activation, since what is desired is a rapid virus-induced memory B cell response after transmission. To determine if PBMC-derived or tonsil leukocyte-derived MP could be suppressive for memory B cell activation, a memory B cell Ig induction assay was used using pokeweed mitogen (PWM)+class B oCpG (Crotty et al, J. Immunol. Methods 286:111-122 (2004)). The addition of MP in PWM-stimulated tonsil cell cultures reduced total IgG and IgA production by 70.8%+/−SEM for IgG (p=0.0064) and 94.2%+/−SEM for IgA (p=0.00004) (FIG. 24A); B cell suppression by MPs was dose-dependent (FIG. 24B). Similar results were observed when MP were generated from autologous tonsil leukocytes or from the Jurkat T cell line (data not shown).

In summary, a major finding in this study is the early appearance of a peak of TRAIL at 17 days of transmission in plasma donors, and implies the TRAIL/DR5 in a key pathway in HIV-1 induced cell death immediately following transmission. An IFN-α, TRAIL, DR5 pathway of CD4+ T cell apoptosis has been proposed for chronic HIV-1 infection based on in vitro studies and on studies in HIV-1+ progressor tonsillar tissues (Lum et al, J. Virol. 75:11128-11136 (2001), Herbeuval et al, Clin. Immunol. 123:121-128 (2007), Herbeuvel et al, Blood 106:3524-3531 (2005)). CD4+ T cells in infected subjects are more sensitive to TRAIL-mediated apoptosis than are CD4+ T cells from uninfected subjects due to upregulated TRAIL receptor DR5 (Lum et al, J. Virol. 75:11128-11136 (2001), Herbeuval et al, Clin. Immunol. 123:121-128 (2007), Herbeuvel et al, Blood 106:3524-3531 (2005), Jeremias et al, Eur. J. Immunol. 28:143-152 (1998)). In vitro, HIV-1 gp120 (Herveuval et al, Blood 105:2458-2464 (2005)) induces monocyte and plasmacytoid dendritic cell IFN-α, which in turn induces CD4+ T cell and monocyte/macrophage TRAIL (Lum et al, J. Virol. 75:11128-11136 (2001), Herbeuval et al, Clin. Immunol. 123:121-128 (2007), Herbeuvel et al, Blood 106:3524-3531 (2005)). HIV-1 Tat has also been reported to induce TRAIL as a mechanism of bystander killing of CD4+ T cells (Yang et al, J. Virol. 77:6700-6708 (2003)).

An important question is why do plasma TRAIL levels peak earlier after HIV-1 transmission than do plasma Fas ligand, TNFR2 and MP? Plasma elevations of TRAIL, Fas ligand and TNFR2 occur in chronic HIV-1, and can be induced by immune cell activation, cell death, or both (Herveuval et al, Blood 105:2458-2464 (2005), Aukrust et al, J. Infect. Dis. 169:420-424 (1994), Hober et al, Infection 24:213-217 (1996), Hosaka et al, J. Infect. Dis. 178:1030-1039 (1998)). Stacy et al (Stacey et al and the NIAID Centre for HIV/AIDS Vaccine Immunology. Elevations in plasma levels of innate cytokines prior to the peak in plasma viremia in acute HIV-1 infection (2007)) have found a burst of IFN-α in the same plasma donors that coincides with the timing of the TRAIL peak seen in this study. Thus, the plasma TRAIL peak that precedes the VL plasma peak may be due either to early apoptosis, but may also result from immune activation and pDC production of IFN-α in response to rising VL. It is hypothesized that the later appearance of elevated plasma Fas ligand, TNFR2 and microparticles maybe the result of, or in response to, massive cell death, as this peak comes at an analogous time to the cell death peak documented in experimental SIV infection in rhesus macaques (Veazey et al, Science 280:427-431 (1998), Haase, Nat. Rev. Immunol. 5:783-792 (2005), Li et al, Nature 434:1148-1152 (2005), Mattapallil et al, Nature 434:1093-1097 (2005)).

Veazey (Mattapallil et al, Nature 434:1093-1097 (2005)) noted the onset of CD4+ gut T cell loss as early as 7 days after SIV infection. In humans, Guadalupe et al (J. Virol. 77:11708-11717 (2003), Mehandru et al, J. Exp. Med. 200:761-770 (2004) and Mehandru and colleagues (Brenchley et al, J. Exp. Med. 200:749-759 (2004)) have studied 2, 1 and 9 patients, respectively, during the first month of HIV-1 infection and found depletion of gut CD4+ T cells.

The eclipse phase of HIV-1 infection is the time from transmission until the appearance of plasma viremia, and is estimated to be 10 days with a range of 7-21 days (Clark et al, N. Engl. J. Med. 324:954-960 (1991), Gaines et al, BMJ 297:1363-1368 (1988), Littl et al, J. Exp. Med. 190:841-850 (1999), Schacker et al, Ann. Intern. Med. 125:257-264 (1996)). The time from appearance of HIV-1 viremia until the first antibody response and symptomatic HIV-1 infection (and therefore establishment of the latent pool) is approximately 14 days (Cooper et al, J. Infect. Dis. 155:1113-1118 (1987), Daar et al, N. Engl. J. med. 324:961-964 (1991), Gaines et al, Lancet 1:1249-1253 (1987)). Thus, the maximal window of opportunity for preventive HIV-1 vaccine efficacy without cell death-induced immune suppression is approximately 24 days. With mediators of apoptosis and immune suppression present as early as day 17 following transmission (10 days average eclipse phase+onset of TRAIL 7 days after T₀), the window of opportunity is narrowed to ˜14-17 days.

The presence of TRAIL, TNFR2 and elevated MP during this early period of acute HIV-1 infection suggests at least four potential mechanisms of immunosuppression. First, direct HIV-1 infection results in loss of a substantial proportion of CD4+ T cells, although the numbers of infected cells does not account for all CD4+ T cell depletion ((Guadalupe et al, J. Virol. 77:11708-11717 (2003), Brenchley et al, J. Exp. Med. 200:749-759 (2004), Mehandru et al, J. Exp. Med. 200:761-770 (2004), Fiebig et al, AIDS17:1871-1879 (2003)). Second, in uninfected CD4+ T cells, TRAIL induces bystander killing ((Lum et al, J. Virol. 75:11128-11136 (2001), Herbeuval et al, Clin. Immunol. 123:121-128 (2007), Herveuval et al, Blood 105:2458-2464 (2005)). In this regard, Miura et al (J. Exp. Med. 193:651-660 (2001)) have shown that administration of an anti-TRAIL mAb in HIV-1 infected hu-PBL-NOD-SCID mice markedly reduces CD4+ T cell apoptosis.

Third, suppression of immune responses can be mediated by T cell MP (Huang et al, J. Immunol. 177:2304-1313 (2006), Distler et al, Arth. Rheum. 52:33337-3348 (2005), Krysko et al, Apoptosis 11:1709-1726 (2006)). CXCR4+ and CCR5+ MP can transfer co-receptors to co-receptor negative cells making them susceptible to HIV-1 (Mack et al, Nat. Med. 6:769-775 (2000), Rozmyslowicz et al, AIDS17:33-42 (2003)). Phagocytosis of MP by macrophages releases TGF-βprostaglandin E2 and IL-10 that can inhibit antigen-specific T and B cell responses (Huang et al, J. Immunol. 177:2304-1313 (2006), Hoffmann et al, J. Immunol. 174:1393-1404 (2005), Huynh et al, J. Clin. Invest. 109:41-50 (2002)). In this regard, Estes et al (J. Infect. Dis. 193:703-712 (2006)). have demonstrated dramatic increases in lymph node TGF-β and IL-10 on day 12 following SW infection. Importantly, it has been directly shown that PBMC and tonsillar cell MP can directly inhibit memory B cell activation (FIG. 24).

Finally, both Fas ligand and TRAIL are incorporated into MP (Huynh et al, J. Clin. Invest. 109:41-50 (2002), Koppler et al, Eur. J. Immunol. 36:648-660 (2006), Crotty et al, J. Immunol. Methods 286:111-122 (2004)). Fas ligand expressing MP can directly induce apoptosis in nearby cells (Huang et al, J. Immunol. 177:2304-1313 (2006), Jodo et al, J. Biol. Chem. 276:39938-39944 (2001), Monleon et al, J. Immunol. 167:6736-6744 (2001)) activated T cells can be the target of Fas ligand mediated proapoptotic microvesicles (Monleon et al, J. Immunol. 167:6736-6744 (2001)). Salvato et al (Clinical and Developmental Immunology (2008)) have recently suggested that treatment of SW-infected macaques with a mAb against Fas ligand attenuates disease and may lead to elevated antibody responses to SW.

Thus, the production of high levels of biologically active plasma mediators and byproducts of cell death during the first two to three weeks of HIV-1 transmission raises the notion that the window of opportunity for a preventive vaccine to work may be shorter than previously thought, ie within the first 14-17 days of transmission, placing considerable constraints on the time available for development of robust anti-HIV-1 immunity following transmission. Preventive vaccine candidates may need to target HIV-1 molecules that induce cell death and be designed to induce protective immune responses to HIV-1 that will either be at maximum inhibitory levels at the time of transmission, or be boosted within hours to days as a secondary immune response to extinguish HIV-1 before HIV-1-induced immunosuppression occurs.

Inhibition of cell death and immunosuppressive MP mediated effects by a vaccine for HIV or other infectious agents may be important as well. This could be accomplished, for example, by an HIV vaccine component inducing anti-lipid antibodies or antibodies against other components of microparticles to facilitate clearance of microparticles and/or to block microparticle toxic effects.

Another use of the data herein is as a rationale for the treatment of HIV-1. For example, antibodies against TNFR or TNF-α, antiphosphatidylserine antibodies or other inhibitors of cell death (Fas-Fc as an inhibitor of FAS-FAS ligand interactions and DR5-Fc as an inhibitor of TRAIL DR5 interactions) can be used to inhibit cell death in HIV as a therapy.

All documents and other information sources cited above are hereby incorporated in their entirety by reference.

What is claimed is:

1. A method of identifying a patient infected with human immunodeficiency virus (HIV) that is in need of treatment with an anti-HIV drug, which method comprises:
i) obtaining a plasma sample from said patient, and
ii) determining the level of Fas ligand, tumor necrosis factor receptor 2 (TNFR2), microparticles or TRAIL in said sample,
wherein a patient having an elevated plasma level of Fas ligand, TNFR2, microparticles or TRAIL, relative to a control, is a patient in need of said treatment.

2. The method according to claim 1 wherein said control is the plasma level of Fas ligand, TNFR2, microparticles or TRAIL in said patient prior to infection with HIV or in an uninfected patient.

3. The method according to claim 1 wherein said patient in need of said treatment has a plasma level of Fas ligand, TNFR2, microparticles or TRAIL at least twice that of said control.

4. The method according to claim 1 wherein the level of microparticles is determined.

5. The method according to claim 1 wherein the level of microparticles is determined by flow cytometry or microparticle capture on antibody-coated plates.

6. The method according to claim 1 wherein said microparticles are CD45+, phosphatidylserine+ or CCR5+.

7. The method according to claim 1 wherein the level of Fas ligand is determined.

8. The method according to claim 1 wherein the level of Fas ligand is determined by an ELIZA assay.

9. The method according to claim 1 wherein the level of TNFR2 is determined.

10. The method according to claim 1 wherein the level of TNFR2 is determined by an ELIZA assay.

11. The method according to claim 1 wherein the level of TRAIL is determined.

12. The method according to claim 1 wherein the level of TRAIL is determined by an ELIZA assay.

13. The method according to claim 1 wherein said identification is effected during acute HIV infection.

14. A method of detecting immune system destruction in a patient infected with HIV comprising:
i) obtaining a plasma sample from said patient, and
ii) determining the level of microparticles in said sample, wherein said microparticles are phosphatidylserine+, CCR5+, CD3+ or CD19+,
wherein a patient having an elevated plasma level of said microparticles, relative to a control, is a patient suffering from immune system destruction.

15. The method according to claim 14 wherein said control is the plasma level of microparticles in said patient prior to infection with HIV or in an uninfected patient.

16. The method according to claim 14 wherein said patient suffering from immune system destruction has a plasma level of microparticles at least twice that of said control.

17. The method according to claim 14 wherein said microparticles are CD45+.

18. The method according to claim 14 further comprising determining the level of Fas ligand, TNFR2 or TRAIL in said sample, wherein an elevated level of Fas ligand, TNFR2 or TRAIL, relative to a control, is further indicative of immune system destruction in said patient.
